SEQUENCE LISTING (28 sequences)

SEQ ID NO: 1 - DNA, 381 bp, Artificial Sequence
  Nucleotide acid sequence of Variable region of murine 3F8 light chain
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggcacttttaaactcctgatctactacacatcaatattacactcaggagtcccatcaaggttcagtggcagtgggtctggaacagattattctctcaccattagcaacctggagcaagaggatattgccacttacttttgccaacagggtgatacgcttcctcccacgttcggtgctgggaccaagctggagctgaaa

SEQ ID NO: 2 - protein, 127 aa, Artificial Sequence
  Amino acid sequence of Variable region of murine 3F8 light chain
MMSSAQFLGLLLLCFQGTRCDIQMTQTTSSLSASLGDRVTISCRASQDISNYLNWYQQKPDGTFKLLIYYTSILHSGVPSRFSGSGSGTDYSLTISNLEQEDIATYFCQQGDTLPPTFGAGTKLELK

SEQ ID NO: 3 - protein, 20 aa, Artificial Sequence
  Leader of Variable region of murine 3F8 light chain
MMSSAQFLGLLLLCFQGTRC

SEQ ID NO: 4 - protein, 23 aa, Artificial Sequence
  LFR1 of Variable region of murine 3F8 light chain
DIQMTQTTSSLSASLGDRVTISC

SEQ ID NO: 5 - protein, 11 aa, Artificial Sequence
  CDR-L1 of Variable region of murine 3F8 light chain
RASQDISNYLN

SEQ ID NO: 6 - protein, 15 aa, Artificial Sequence
  LFR2 of Variable region of murine 3F8 light chain
WYQQKPDGTFKLLIY

SEQ ID NO: 7 - protein, 7 aa, Artificial Sequence
  CDR-L2 of Variable region of murine 3F8 light chain
YTSILHS

SEQ ID NO: 8 - protein, 32 aa, Artificial Sequence
  LFR3 of Variable region of murine 3F8 light chain
GVPSRFSGSGSGTDYSLTISNLEQEDIATYFC

SEQ ID NO: 9 - protein, 9 aa, Artificial Sequence
  CDR-L3 of Variable region of murine 3F8 light chain
QQGDTLPPT

SEQ ID NO: 10 - protein, 11 aa, Artificial Sequence
  LFR4 of Variable region of murine 3F8 light chain
FGAGTKLELKR

SEQ ID NO: 11 - DNA, 408 bp, Artificial Sequence
  Nucleotide sequence of Variable region of murine 3F8 heavy chain
atgaaagtgttgagtctgttgtacctgttgacagccattcctggtatcctgtctgatgtacaacttcaggagtcaggacctggcctcgtgaaaccttctcagtctctgtctctcacctgctctgtcactggctactccatcaccagtgcttattactggaactggatccggcagtttccaggagacaaactggaatggatgggctacataagctacgacggtcgcaataatttcaacccatctctcaaaaatcgaatctccatcactcgtgacacatctaagaaccagtttttcctgaagttgaattctgtgacttctggggacacagctacatattactgtgcaagagatggggattacgactactttgactactggggccaaggcaccactctcacagtctcctca

SEQ ID NO: 12 - protein, 136 aa, Artificial Sequence
  Amino acid sequence of Variable region of murine 3F8 heavy chain
MKVLSLLYLLTAIPGILSDVQLQESGPGLVKPSQSLSLTCSVTGYSITSAYYWNWIRQFPGDKLEWMGYISYDGRNNFNPSLKNRISITRDTSKNQFFLKLNSVTSGDTATYYCARDGDYDYFDYWGQGTTLTVSS

SEQ ID NO: 13 - protein, 18 aa, Artificial Sequence
  Leader  of Variable region of murine 3F8 heavy chain
MKVLSLLYLLTAIPGILS

SEQ ID NO: 14 - protein, 30 aa, Artificial Sequence
  HFR1 of Variable region of murine 3F8 heavy chain
DVQLQESGPGLVKPSQSLSLTCSVTGYSIT

SEQ ID NO: 15 - protein, 6 aa, Artificial Sequence
  CDR-H1 of Variable region of murine 3F8 heavy chain
SAYYWN

SEQ ID NO: 16 - protein, 14 aa, Artificial Sequence
  HFR2 of Variable region of murine 3F8 heavy chain
WIRQFPGDKLEWMG

SEQ ID NO: 17 - protein, 16 aa, Artificial Sequence
  CDR-H2 of Variable region of murine 3F8 heavy chain
YISYDGRNNFNPSLKN

SEQ ID NO: 18 - protein, 32 aa, Artificial Sequence
  HFR3 of Variable region of murine 3F8 heavy chain
RISITRDTSKNQFFLKLNSVTSGDTATYYCAR

SEQ ID NO: 19 - protein, 9 aa, Artificial Sequence
  CDR-H3 of Variable region of murine 3F8 heavy chain
DGDYDYFDY

SEQ ID NO: 20 - protein, 11 aa, Artificial Sequence
  HFR4 of Variable region of murine 3F8 heavy chain
WGQGTTLTVSS

SEQ ID NO: 21 - protein, 467 aa, Artificial Sequence
  Amino acid sequence of humanized 3F8-heavy chain of Clone 3
MGWSCIILFLVATATGVHSQVQLQESGPGLVKPSETLSLTCTVSGYSITSAYYWNWIRQPFGKGLEWMGYISYDGRNNFNPSLKNRVSISRDTSKNQFSLKLSSVTAADTATYYCARDGDYDYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 1407 bp, Artificial Sequence
  Nucleotide acid sequence of humanized 3F8-heavy chain of Clone 3
atgggctggtcatgcattattctgtttctggtcgcaactgctacaggcgtgcatagtcaagtgcagctgcaggagagcggacctggcctcgtgaagccaagcgagacactgtctctgacatgtaccgtgtccggctacagcatcacctccgcctactactggaactggatccggcagccttttggcaagggcctggaatggatgggctacatcagctacgacggcagaaacaacttcaaccccagcctgaaaaatagagtgtccatctctcgggacaccagcaagaaccagttcagcctgaagctgagcagcgtgacagccgctgataccgccacatactactgcgccagagacggagattatgactacttcgactactggggccagggcaccaccgtcaccgtgtctagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 23 - protein, 233 aa, Artificial Sequence
  Amino acid sequence of humanized 3F8-light chain of Clones 1-3
MGWSCIILFLVATATGVHSDIQMTQSPSSLSASVGDRVTITCRASQDISNYLNWYQQKPGKAPKLLIYYTSILHSGVPSRFSGSGSGTDYTFTISSLQPEDIATYFCQQGDTLPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 705 bp, Artificial Sequence
  Nucleotide acid sequence of humanized 3F8-light chain of Clones 1-3
atgggctggtcatgtattattctgtttctggtcgcaactgctacaggggtccatagtgatattcagatgacccagagccccagcagcctgagcgccagcgtgggcgatagagtgaccatcacatgtcgggcctctcaggacatcagcaactacctgaactggtatcagcaaaagcccggcaaagcccctaagctgctgatctactacaccagcatcctgcacagcggagtgccatctagattcagcggctctggcagcggcaccgactacacatttaccatctcctccctccagcctgaggacatcgctacatacttctgccagcagggcgacaccctgcctcctaccttcggcggcggaacaaagctggaaatcaagaggacagtggccgccccaagcgtgttcatctttcccccttccgacgagcagctgaagtctggcaccgccagcgtggtgtgcctgctgaacaacttctaccctcgggaggccaaggtccagtggaaggtggataacgccctgcagtctggcaatagccaggagtccgtgaccgagcaggactctaaggatagcacatattccctgtctagcaccctgacactgagcaaggccgattacgagaagcacaaggtgtatgcctgtgaagtcacccatcaggggctgtcatcacccgtcactaagtcattcaatcgcggagaatgctgataa

SEQ ID NO: 25 - protein, 467 aa, Artificial Sequence
  Amino acid sequence of humanized 3F8-heavy chain of Clone 1
MGWSCIILFLVATATGVHSQVQLQESGPGLVKPSETLSLTCTVSGYSITSAYYWNWIRQPPGKGLEWIGYISYDGRNNFNPSLKNRVTISVDTSKNQFSLKLSSVTAADTAVYYCARDGDYDYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1407 bp, Artificial Sequence
  Nucleotide acid sequence of humanized 3F8-heavy chain of Clone 1
atgggctggtcatgcattattctgtttctggtcgcaactgctacaggcgtgcatagtcaagtgcagctgcaggagagcggccctggactggtgaagcctagcgagacactgtctctcacctgtaccgtgtctggctacagcatcacctccgcctactactggaactggatccggcagcctccaggcaagggcctggaatggatcggctacatcagctacgacggcagaaacaacttcaaccccagcctgaaaaatagagtgaccatctctgtggacaccagcaagaaccagtttagcctgaagctgagcagcgtgacagccgctgataccgccgtgtactactgcgccagagacggagattatgactacttcgactactggggccagggcaccacagtcacagtgtccagcgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

SEQ ID NO: 27 - protein, 467 aa, Artificial Sequence
  Amino acid sequence of humanized 3F8-heavy chain of Clone 2
MGWSCIILFLVATATGVHSQVQLQESGPGLVKPSETLSLTCTVSGYSITSAYYWNWIRQPFGKGLEWMGYISYDGRNNFNPSLKNRVTISRDTSKNQFSLKLSSVTAADTAVYYCARDGDYDYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1407 bp, Artificial Sequence
  Nucleotide acid sequence of humanized 3F8-heavy chain of Clone 2
atgggctggtcatgcattattctgtttctggtcgcaactgctacaggcgtgcatagtcaagtgcagctgcaggagagcggccccggcctggtgaagcctagcgagacactgagcctcacctgtaccgtgtccggctacagcatcaccagcgcctactactggaactggatccggcagccttttggcaagggcctggaatggatgggctacatctcctacgacggcagaaacaacttcaacccatctctgaaaaatagagtgaccatcagccgggacacaagcaagaaccagttcagcctgaagctgtctagcgtgacagccgctgataccgccgtgtactactgcgccagagacggagattatgactacttcgactactggggacagggcaccaccgtgacagtcagctctgccagcaccaagggcccttccgtgtttccactggccccctcctctaaatccacatctggcggcaccgccgccctgggctgtctggtgaaggactacttcccagagcctgtgacagtgtcctggaactctggcgccctgacatccggcgtgcacacatttccagccgtgctgcagagctccggcctgtacagcctgtctagcgtggtgacagtgccctcctctagcctgggcacacagacctatatctgcaacgtgaatcacaagccaagcaataccaaggtggacaagaaggtggagcccaagtcctgtgataagacacacacctgccccccttgtcctgctcccgagctgctgggcggccctagcgtgttcctgtttccacccaagcctaaggacaccctgatgatctcccggacacccgaggtgacctgcgtggtggtggacgtgtctcacgaggatcctgaggtgaagttcaactggtatgtggatggcgtggaggtgcacaatgccaagaccaagcccagagaggagcagtacaactctacatatagggtggtgagcgtgctgaccgtgctgcaccaggactggctgaacggcaaggagtataagtgcaaggtgtccaataaggccctgcccgcccccatcgagaagacaatcagcaaggccaagggccagcctcgggagccacaggtgtacaccctgcctccatccagagacgagctgacaaagaaccaggtgtctctgacatgtctggtgaagggcttctatcctagcgatatcgccgtggagtgggagtccaatggccagccagagaacaattacaagaccacaccccctgtgctggactccgatggctccttctttctgtattccaagctgaccgtggataagtctcggtggcagcagggcaacgtgttcagctgttccgtgatgcacgaagccctgcataatcactatactcagaaatccctgtccctgtcacctggaaagtgataa

(19) United States
(12) Patent Application Publication
(10) Pub. No.: US 2024/0293565 A1
(43) Pub. Date: Sep. 5, 2024

(54) ANTI-HER3 ANTIBODY, ANTIBODY DRUG CONJUGATE CONTAINING THE SAME, AND USE THEREOF

(71) Applicant: BEIJING SINOTAU BIO-PHARMACEUTICALS TECHNOLOGY CO., LTD., Beijing (Daxing District) (CN)

(72) Inventors: Xiaoyan ZHONG, Beijing (CN); Zhe Li, Beijing (CN); Jie ZHU, Beijing (CN)

(73) Assignee: BEIJING SINOTAU BIO-PHARMACEUTICALS TECHNOLOGY CO., LTD., Beijing (Daxing District) (CN)

(21) Appl. No.: 18/570,403

(22) PCT Filed: Jun. 15, 2022
(86) PCT No.: PCT/CN2022/098929

§ 371 Date: Dec. 14, 2023

(30) Foreign Application Priority Data
Jun. 15, 2021 (WO) PCT/CN2021/099998

(51) Int. Cl. A61K 47/68; A61K 35/00; C07K 16/32

(52) U.S. Cl. CPC A61K 47/68031; A61K 35/00; A61K 47/6849; A61K 47/6889; C07K 16/32; C07K 2317/24; C07K 2317/565; C07K 2317/76

(57) ABSTRACT

Provided is an anti-HER3 antibody, or an antibody drug conjugate (ADC) containing the anti-HER3 antibody. Also provided is the use of the antibody or ADC in the treatment of HER3-expression cancers.

### CROSS REFERENCE TO RELATED APPLICATIONS

This application claims priority from PCT international application PCT/CN 2021/099998 filed on Jun. 15, 2021, which is incorporated herein by reference in its entirety.

### FIELD OF THE INVENTION

The present disclosure relates to an anti-HER3 antibody. The present disclosure also relates to an antibody drug conjugate containing the anti-HER3 antibody and their uses in the treatment of HER3-expression cancers.

### BACKGROUND

HER3 is a member of the ERBB family, which plays key role in the cell proliferation, tumor metastasis and drug resistance. While drugs targeting EGFR and HER2 demonstrate great clinical benefits in alleviating multiple cancers, previous endeavour of developing anti-HER3 antibodies for cancer therapy fell short, repeatedly, suggesting that just tackling HER3 alone and the pathway it dangles around may not be sufficient enough in inhibiting tumor growth. Consistent with this hypothesis, U3-1402, a HER3 targeting ADC, has demonstrated a promising outcome from an early phase clinical trial on breast and NSCLC. Moreover, a bispecific antibody that manipulates both HER3 and HER2 significantly reduces disease biomarker in some enriched population. The most recent clinical advancement suggests that HER3 remains a promising oncology target, if with additional add-on mechanism.

### SUMMARY

In one aspect, the present disclosure provides an anti-HER3 antibody or an antigen-binding fragment thereof, comprising a heavy chain variable region (VH), wherein the VH comprise: CDR-H1 comprising the amino acid sequence of SEQ ID NO: 15, CDR-H2 comprising the amino acid sequence of SEQ ID NO: 17 and CDR-H3 comprising the amino acid sequence of SEQ ID NO: 19.

In some embodiments, the VH comprise CDR-H1 of SEQ ID NO: 15, CDR-H2 of SEQ ID NO: 17 and CDR-H3 of SEQ ID NO: 19.

In some embodiments, the anti-HER3 antibody or antigen-binding fragment thereof further comprises a light chain variable region (VL), wherein the VL comprises: CDR-L1 comprising the amino acid sequence of SEQ ID NO: 5, CDR-L2 comprising the amino acid sequence of SEQ ID NO: 7 and CDR-L3 comprising the amino acid sequence of SEQ ID NO: 9.

In some embodiments, the VL comprises CDR-L1 of SEQ ID NO: 5, CDR-L2 of SEQ ID NO: 7 and CDR-L3 of SEQ ID NO: 9.

In some embodiments, the VH comprises the amino acid sequence of SEQ ID NO: 12 with or without a leader sequence of SEQ ID NO: 13, or an amino acid sequence at least 85%, 86%, 87%, 88%, 89%, 90%, 91%, 92%, 93%, 94%, 95%, 96%, 97%, 98%, or 99% identical thereto.

In some embodiments, the VL comprises the amino acid sequence of SEQ ID NO: 2 with or without a leader sequence of SEQ ID NO: 3, or an amino acid sequence at least 85%, 86%, 87%, 88%, 89%, 90%, 91%, 92%, 93%, 94%, 95%, 96%, 97%, 98%, or 99% identical thereto.

In some embodiments, the anti-HER3 antibody is a mouse, chimeric, humanized or human antibody.

In some embodiments, the heavy chain of the anti-HER3 antibody is of the type of IgG1.

In some embodiments, the anti-HER3 antibody is a humanized antibody, and the heavy chain comprises the amino acid sequence of SEQ ID NO: 21, 25 or 27, or an amino acid sequence at least 85%, 86%, 87%, 88%, 89%, 90%, 91%, 92%, 93%, 94%, 95%, 96%, 97%, 98%, or 99% identical thereto.

In some embodiments, the anti-HER3 antibody is a humanized antibody, and the light chain comprises the amino acid sequence of SEQ ID NO: 23, or an amino acid sequence at least 85%, 86%, 87%, 88%, 89%, 90%, 91%, 92%, 93%, 94%, 95%, 96%, 97%, 98%, or 99% identical thereto.

In some embodiments, the HER3 is human or monkey HER3.

In some embodiments, the anti-HER3 antibody blocks NRG1 induced phosphorylation of the HER3.

In some embodiments, the anti-HER3 antibody binds to human or monkey HER3 with an EC₅₀ lower than 1 nM.

In some embodiments, the anti-HER3 antibody has an internalization activity upon binding to the HER3.

In another aspect, the present disclosure provides an isolated nucleic acid comprising a polynucleotide acid sequence encoding a VH described above and/or a VL described above.

In some embodiments, the isolated nucleic acid is selected from SEQ ID NOs: 1, 11, 22, 24, 26 and 28.

In another aspect, the present disclosure provides a vector comprising the isolated nucleic acid.

In another aspect, the present disclosure provides a host cell comprising the isolated nucleic acid or the vector.

In another aspect, the present disclosure provides a host cell expressing the anti-HER3 antibody or antigen-binding fragment thereof.

In another aspect, the present disclosure provides an antibody conjugate comprising the anti-HER3 antibody or antigen-binding fragment thereof conjugated to a chemical moiety.

In some embodiments, the anti-HER3 antibody or antigen-binding fragment thereof is conjugated to the chemical moiety via a linker

In some embodiments, the linker is enzyme-cleavable.

In some embodiments, the linker comprises a Val-Cit moiety.

In some embodiments, the antibody conjugate is an antibody drug conjugate (ADC).

In some embodiments, the chemical moiety is a radioactive isotope, a chemotherapeutic agent, or a cytotoxic agent.

In some embodiments, the cytotoxic agent is a toxin.

In some embodiments, the toxin is selected from auristatin E, auristatin F, MMAE and MMAF.

In another aspect, the present disclosure provides a pharmaceutical composition comprising the anti-HER3 antibody or antigen-binding fragment thereof or the antibody conjugate and a pharmaceutically acceptable carrier.

In some embodiments, the pharmaceutical composition further comprises one or more other anti-cancer agents.

In another aspect, the present disclosure provides uses of the anti-HER3 antibody or antigen-binding fragment thereof or the antibody conjugate in the manufacture of a medicament for the treatment of a cancer.

In some embodiments, the cancer expresses the HER3.

In some embodiments, the cancer is a gastric or colorectal cancer.

In another aspect, the present disclosure provides a method of treating a cancer in a subject, comprising administrating to the subject a therapeutically effective amount of the anti-HER3 antibody or antigen-binding fragment thereof, the antibody conjugate, or the pharmaceutical composition.

In some embodiments, the cancer expresses the HER3.

In some embodiments, the cancer is a gastric or colorectal cancer.

### BRIEF DESCRIPTION OF THE DRAWINGS

FIG. 1 shows the murine 3F8 specifically binds SP2/0-HER3 cells.

FIG. 2 shows the binding affinity of murine 3F8 with human HER3, HER2 and EGFR as determined by ELISA.

FIG. 3 shows the murine 3F8 recognizes both human and monkey HER3 with similar potency as determined by ELISA.

FIG. 4 shows murine 3F8 blocks NRG1-induced phosphorylated HER3.

FIG. 5 shows murine 3F8 is rapidly up taken by cells of varying surface level of HER3.

FIG. 6 shows anti-HER3 antibodies efficiently inhibit tumor growth in the BT474 subcutaneous xenograft model.

FIG. 7 shows [⁸⁹Zr]Zr-ch3F8 imaging of gastric PDX model GAS078.

FIG. 8 shows representative [⁸⁹Zr]Zr-ch3F8 imaging in 6 PDX models.

FIG. 9 shows ch3F8-MMAE maintains the similar binding affinity to ch3F8.

FIG. 10 shows cytotoxicity of ch3F8-MMAE in multiple cell lines.

FIG. 11 shows ch3F8-MMAE inhibits tumor growth in the gastric model GAS078.

FIGS. 12A-12C show hu3F8 maintains binding affinity after stress test of heat, acid and repeat free-thaw. FIG. 12A, three clones of hu3F8 were incubated at pH3.5 for 0, 2, 4 and 6 hours and then proceeded to ELISA assay to measure the binding affinity. FIG. 12B, three clones of hu3F8 were incubated at 40° C. for varying days and then proceeded to ELISA assay to measure the binding affinity. FIG. 12C, three clones of hu3F8 were frozen and thaw for 3 or 5 cycles and then proceeded to ELISA assay to measure the binding affinity.

FIGS. 13A-13B show hu3F8-MMAE dose-dependently inhibits tumor growth (FIG. 13A) and has little impact on the body weight (FIG. 13B).

FIG. 14 shows hu3F8-MMAE inhibits tumor growth in the gastric PDX model GAS078 at 10 mg/kg with a single shot.

FIG. 15 shows hu3F8-MMAE inhibits tumor growth in the gastric PDX model GAS078 at 6 mg/kg.

FIG. 16 shows hu3F8-MMAE inhibits tumor growth in the colorectal PDX model CS226.

### DETAILED DESCRIPTION

Unless defined otherwise, technical and scientific terms used herein have the same meaning as commonly understood by a person of ordinary skill in the art. Any methods, devices and materials similar or equivalent to those described herein can be used in the practice of the present invention. The following definitions are provided to facilitate understanding of certain terms used herein and are not meant to limit the scope of the present disclosure.

The articles “a” and “an” are used herein to refer to one or to more than one (i.e., to at least one) of the grammatical object of the article. By way of example, “an element” means one element or more than one element.

The term “and/or” used herein is to be taken as specific disclosure of each of the two specified features or components with or without the other. Thus, the term “and/or” as used in a phrase such as “A and/or B” is intended to include “A and B,” “A or B.” “A” (alone), and “B” (alone).

“Human epidermal growth factor receptor 3 (HER3)”, also called receptor tyrosine-protein kinase erbB-3 (ERBB3), is a member of the EGFR/ERBB family. Unlike other ERBB family members HER2 and EGFR, HER3 itself bears no kinase activity. Accordingly, HER3 has to associate with its kinase active members, either EGFR or HER2, as a heterodimer to trigger its downstream activity. Upon binding to its native ligand NRG1, HER3 goes through conformation change, heterodimerization and phosphorylation, followed by signal transduction in activating MAPK, PI3K/Akt and PLCγ. At the meantime, HER3 also implements its biological activity via ligand independent way at the presence of high level HER2. HER3 plays a key role in cell growth and proliferation, embryonic development and oncogenesis. HER3 knockout mice are severely underdeveloped and lethal at embryonic day 13.5. HER3 also contributes to drug resistance of drugs targeting varying proteins and indications.

The term “antibody” generally refers to any immunoglobulin (Ig) molecule comprised of four polypeptide chains, two heavy (H) chains and two light (L) chains, or any functional fragment (antigen-binding fragment) thereof, which retains the essential epitope binding features of the Ig molecule. In a full-length antibody, each heavy chain is comprised of a heavy chain variable region (abbreviated herein as HCVR or VH) and a heavy chain constant region. The heavy chain constant region is generally comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The CDRs of a heavy chain are therefore referred to as CDR-H1, CDR-H2 and CDR-H3, respectively, from the amino-terminal side of the heavy chain, whereas the CDRs of a light chain are referred to as CDR-L1, CDR-L2 and CDRL3, respectively, from the amino-terminal side of the light chain. Immunoglobulin molecules can be of any type (e.g., IgG, IgE, IgM, IgD, IgA and IgY), class (e.g., IgG 1, IgG2, IgG 3, IgG4, IgA1 and IgA2) or subclass. In a broad meaning, the term “antibody” further refers to scFv or sdAb which is not derived from an immunoglobulin molecule with four polypeptide chains. The term “antibody” further refers to any multi-specific antibody (especially bispecific antibody) containing the anti-HER3 antibody or an antigen-binding fragment thereof.

An antibody or functional fragment of the antibody may have one or more modified amino acid residues. For example, the heavy chain or the light chain of the antibody has undergone one or two or more modifications selected from the group consisting of N-linked glycosylation, O-linked glycosylation, N-terminal processing, C-terminal processing, deamidation, isomerization of aspartic acid, oxidation of methionine, addition of a methionine residue to the N-terminus, amidation of a proline residue, conversion of N-terminal glutamine or N-terminal glutamic acid to pyroglutamic acid, and a deletion of one or two amino acids from the carboxyl terminus.

An “antigen-binding fragment” is a portion of an antibody, for example as F(ab′)2, Fab, Fv, scFv, sdAb, and the like. An antigen-binding fragment of a full length antibody retains the target specificity of a full length antibody. Recombinant functional antibody fragments, such as scFv (single chain variable chain fragments), have therefore been used to develop therapeutics as an alternative to therapeutics based on mAbs. scFv fragments (˜25 kDa) consist of the two variable domains, VH and VL. Naturally, VH and VL domains are non-covalently associated via hydrophobic interaction and tend to dissociate. However, stable fragments can be engineered by linking the domains with a hydrophilic flexible linker to create a scFv.

As used herein, the term “single domain antibody” (sdAb) has its general meaning in the art and refers to the single heavy chain variable domain of antibodies of the type that can be found in Camelid mammals and are naturally devoid of light chains. Such single-domain antibody is also called V_{H}H or “Nanobody”. The amino acid sequence and structure of a single-domain antibody can be considered to be comprised of four framework regions (FR1, FR2, FR3, and FR4), and three complementary determining regions (CDR1, CDR2, and CDR3). Accordingly, the single-domain antibody can be defined as an amino acid sequence with the general structure: FR1-CDR1-FR2-CDR2-FR3-CDR3-FR4, which is similar to variable domain VH or VL. The use of sdAbs as single antigen-binding proteins or as an antigen-binding domain in larger proteins or polypeptides offer a number of significant advantages over the use of conventional antibodies or other antibody fragments (e.g., scFv). The advantages of sdAbs include: only a single domain is required to bind an antigen with high affinity and with high selectivity; sdAbs are highly stable to denaturing agents or conditions including heat, pH, and proteases; and sdAbs can access targets and epitopes not accessible to conventional antibodies. Typically, sdAbs are produced in camelids such as llamas, but can also be synthetically generated using techniques that are well known in the art.

The term “chimeric antibody” refers to an antibody comprising a variable region, i.e., binding region, from mouse and at least a portion of a constant region derived from a different source or species (e.g., human), usually prepared by recombinant DNA techniques. Chimeric antibodies comprising a mouse variable region and a human constant region are especially preferred. Such mouse/human chimeric antibodies are usually the product of expressed immunoglobulin genes comprising DNA segments encoding mouse immunoglobulin variable regions and DNA segments encoding human immunoglobulin constant regions. Methods for producing chimeric antibodies involve conventional recombinant DNA and gene transfection techniques now well known in the art.

The term “humanized antibody” refers to antibodies in which the framework or “complementarity determining regions” (CDR) have been modified to comprise the CDRs of an immunoglobulin of different specificity as compared to that of the parent immunoglobulin. In a preferred embodiment, the CDRs of the VH and VL are grafted into the framework region of human antibody to prepare the “humanized antibody.” The heavy and light chain variable framework regions can be derived from the same or different human antibody sequences. The human antibody sequences can be the sequences of naturally occurring human antibodies. Optionally the framework region can be modified by further mutations. Particularly preferred CDRs correspond to those representing sequences recognizing the antigens noted above for chimeric antibodies. Preferably such humanized version is chimerized with a human constant region. The term “humanized antibody” as used herein also comprises such antibodies which are modified in the constant region to generate the properties according to the invention, especially in regard to C1q binding and/or FcR binding, e.g. by “class switching”, i.e. change or mutation of Fc parts (e.g. from IgG1 to IgG4 and/or IgG1/IgG4 mutation).

The term “human antibody”, as used herein, is intended to include antibodies having variable and constant regions derived from human germ line immunoglobulin sequences. Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge. Human antibodies can also be produced in phage display libraries.

As used herein, the term “anti-HER3 antibody” refers to an antibody which specifically binds to the human HER3 antigen. An antibody “which specifically binds” an antigen of interest, i.e., HER3, is one capable of binding that antigen with sufficient affinity such that the antibody is useful in targeting a cell expressing the antigen. The binding affinity can be determined with a standard binding assay, such as surface plasmon resonance technique (BIAcore®, GE-Healthcare Uppsala. Sweden).

The term “sequence identity”, with respect to a peptide, or an antibody sequence, is defined as the percentage of amino acid residues in a candidate sequence that are identical with the amino acid residues in a reference peptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “internalization”, when used in relationship with the binding of the antibody of the invention to HER3 antigen at the surface of cancer cells, refers to rapid uptake from the external milieu of the antibody-antigen complex by receptor-mediated endocytosis, micropinocytosis, phagocytosis or other similar cellular uptake and/or trafficking pathways. In one embodiment, “internalization” of the antibody of the invention thus relates to its uptake from the external milieu by a mechanism involving plasma membrane infolding and vesicle formation. When the antibody of the invention is conjugated to chemical moiety, such as a radioactive isotope, a fluorophore or a cytotoxin, the chemical moiety can be internalized into HER3 expressing cells together with the antibody of the present invention. Whether or not an antibody has an internalization activity can be confirmed by a method generally known by those skilled in the art and can be confirmed by, for example a method involving contacting labeling material-bound anti-HER3 antibodies with HER3-expressing cells and confirming whether or not the labeling material (e.g., a radioactive isotope, a fluorophore or a fluorescent protein) is incorporated into the cells, or a method involving contacting cytotoxic substance-conjugated anti-HER3 antibodies with HER3-expressing cells and confirming whether or not the death of the HER3-expressing cells is induced. More specifically, the internalization activity of the anti-HER antibody can be assayed by, for example, a method described in Examples. The anti-HER3 antibody having an internalization activity can be conjugated with, for example, the cytotoxic substance and used as a pharmaceutical composition such as an anticancer agent described later.

The term “host cell” as used herein refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “nucleic acid” as used herein refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.”

The term “isolated nucleic acid” as used herein refers to the purification status, and in such context means the nucleic acid is substantially free of other biological molecules such as, proteins, lipids, carbohydrates, or other material such as cellular debris and growth media.

The term “EC₅₀”, also known as half maximal effective concentration, refers to the concentration of an antibody or an antigen-binding portion thereof that gives half-maximal response, e.g., in a test by FACS or ELISA.

The term “antibody conjugate” as used herein refers to an antibody or an antigen-binding fragment thereof is conjugated to other chemical moiety, such as a radioactive isotope, a chemotherapeutic agent and a toxin. In some embodiments, the chemical moiety is an isotope or a fluorophore, and thus the conjugated antibody can be used to show, through in vivo imaging, cells, tissues or organs (including tumors) which express HER3. In some embodiments, the antibody conjugate is an antibody drug conjugate (ADC).

The terms “anti-HER3 antibody drug conjugate” and “anti-HER3 ADC”, used interchangeably herein, refer to an antibody-drug conjugate comprising an antibody that specifically binds to HER3 and is conjugated via a linker to a cytotoxic agent, e.g., an auristatin. Generally, the antibody (e.g., anti-HER3 antibody) is able to retain its biological activity, such as the binding affinity to its targeted antigen, after modified to be an ADC molecule. The term “cytotoxic agent” refers to a substance that inhibits or prevents the expression activity of cells, function of cells and/or causes destruction of cells. The term is intended to include radioactive isotopes, chemotherapeutic agents, and toxins such as small molecule toxins or enzymatically active toxins of bacterial, fungal, plant or animal origin, including fragments and/or variants thereof. Chemotherapeutic agents are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol), docetaxel, cabazitaxel, and 10-deacetylbaccatin; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozocin (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane and aminoglutethimide; analogs thereof, derivatives thereof, and combinations thereof. Examples of cytotoxic agents include, but are not limited to auristatins (e.g., auristatin E, auristatin F, MMAE and MMAF), auromycins, maytansinoids, ricin, ricin A-chain, combrestatin, duocarmycins, dolastatins, doxorubicin, daunorubicin, taxols, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes. The term “auristatin”, as used herein, refers to a family of antimitotic agents. Auristatin derivatives are also included within the definition of the term “auristatin”. Examples of auristatins include, but are not limited to, auristatin E (AE), monomethyl auristatin E (MMAE), monomethyl auristatin F (MMAF), and synthetic analogs of dolastatin. In one embodiment, the anti-HER3 antibody drug conjugate is anti-HER3 antibody-MMAE (e.g. ch3F8-MMAE or hu3F8-MMAE). In some embodiments, the linker is conjugated to a Cys residue in a hinge region of the antibody.

The term “pharmaceutical composition” refers to a formulation that is in such a form as to permit the biological activity of the active ingredient(s) to be effective and, therefore, may be administered to a subject for therapeutic use.

The term “pharmaceutically acceptable carrier” refers to any inactive substance that is suitable for use in a formulation for the delivery of an active ingredient, such as the antibody or ADC of the present invention. A carrier may be a binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride.

The term “effective amount” or “therapeutically effective amount” refers to the amount of an active agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the dosing regimen to be followed, whether it is administered in combination with other therapeutics, timing of administration, the tissue to be imaged, and the physical delivery system in which it is carried.

The phase “other anti-cancer agent” as used herein refers to an anti-cancer agent different from the anti-HER3 antibody or the anti-HER3 ADC disclosed herein. Non-limiting examples of the other anti-cancer agent include chemotherapeutic agent, such as 5-fluorouracil, hydroxyurea, gemcitabine, methotrexate, doxorubicin, etoposide, carboplatin, cisplatin, cyclophosphamide, melphalan, dacarbazine, taxol, camptothecin, FOLFIRI, FOLFOX, docetaxel, daunorubicin, paclitaxel, oxaliplatin, and combinations thereof; biotherapeutic agent, such as antibodies against PD-L1, PD-1, CTLA-4, CCR4, OX40; ionizing radiation; cellular therapeutics, such as chimeric antigen receptor (CAR) modified T cells or NK cells.

The terms “cancer” and “tumor”, used interchangeably herein, refer to, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymomas, medulloblastomas, meningiomas, squamous cell carcinomas, pituitary adenoma, lymphoma, lymphocytic leukemia, including refractory versions of any of the above cancers, or a combination of one or more of the above cancers. Preferably such cancer is a breast cancer, lung cancer, cancer of the head or neck, or pancreatic cancer, preferably lung cancer, cancer of the head or neck, or pancreatic cancer. Preferably such cancers are further characterized by HER3 expression or overexpression, more preferably by HER3 overexpression.

The term “treating” or “treatment” as used herein means the treating or treatment of a disease or medical condition in a subject, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a subject; (c) suppressing the disease or medical condition, for example by, slowing or arresting the development of the disease or medical condition in a subject; or (d) alleviating a symptom of the disease or medical condition in a subject.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### Material and Method

Materials: The following reagents were purchased from Southern Biotech and used in indicated dilution: goat anti-mouse IgG-HRP (1030-05, 1:5000 dilution), Goat anti-human IgG-PE (2040-09, 1:1000 dilution), Goat anti-Human Kappa IgG-HRP (2061-05, 1:20000 dilution), Goat anti-rabbit IgG-HRP (4030-05, 1:5000 dilution), Streptavidin-FITC (7100-02, 1:500 dilution), Mouse anti-human Kappa-APC (9230-11, 1:500 dilution), Mouse IgG-APC (0107-11, 0.1 mg/ml). NRG1 was from Origene (TP723155). Cell culture medium Roswell Park Memorial Institute (RPMI)1640, Dulbecco's modified eagle's medium (DMEM) and fetal bovine serum were from Hyclone. Recombinant Patritumab was in house prepared. HER3 antibody 3F8 was in house generated from hybridoma or recombination. m3F8 indicates murine 3F8, while ch3F8 and hu3F8 refer to chimeric and humanized 3F8 respectively.

Cell culture: SP2/0, SP2/0-HER3, SP2/0-HER2, SP2/0-EGFR, NCI-N87, MDA-MB-468, MDA-MB-453, 7901, HT29, MCF-7, SK-BR-3 used in the research were purchased from and maintained in the appropriate medium recommended by ATCC. SP2/0-EGFR, SP2/0-HER2, SP2/0-HER3 that stably expressing human EGFR, HER2 and HER3 respectively were generated in house.

Generation of anti-HER3 hybridoma: On day one, BALB/c mice (female, age of 8-10 weeks), were intraperitoneally injected with 1-2×10⁶ SP2/0-HER3 cells that expressing human HER3 together with Freund's complete adjuvant. On day 8, an enhanced immunization was performed with the same amount of cells with Freund's incomplete adjuvant. Since day 14, every three days mice were immunized with the abovementioned amount of cells, and repeated three times. Three days after the last immunization, B lymphocytes from spleens were isolated and fused with immortal myeloma cells NS-1 cells to generate hybridoma cells.

The hybridoma cells were cultured in a 96-well plate, in a series dilution. The supernatants were collected to screen any antibody that recognizing HER3 expressed on the surface of SP2/0 cells by flow cytometry or recombinant HER3 by ELISA.

DNA cloning and sequencing of antibody variable regions: Briefly, total RNA extracted from the hybridomas with Trizol (ThermoFisher) was reversely transcribed into the first cDNA strands. Rapid amplification of 5′ complementary DNA (5′RACE) followed by nested PCR was then adapted to amplify the DNA sequence encoding the variable regions, as described in the instruction of 5′RACE kit (Invitrogen, 18374-058). The PCR products were cloned into pGM-T vector. Positive clones were proceeded for DNA sequencing, from which the protein sequences were deduced accordingly. The amino acids of the variable regions were analyzed in Kabat numbering scheme.

Antibody expression: In short, DNA encoding the antibody heavy chain and light chain were cloned into the expression vector pCDNA3.1(+) (Invitrogen) and expressed in the 293T cells. Antibodies were purified with protein A or G columns (GE).

Humanization: The humanization was performed in GenScript. First, murine-human chimeric antibody (ch3F8) was generated by replacing the constant regions of the heavy chains of the murine antibody with sequences of human IgG1 constant region and replacing the constant regions of the light chains of the murine antibody with sequences of human Ig constant region. Humanization was then processed on top of the chimeric antibody, following the procedure in the reference (Kuramochi et al). The residues in the mouse framework essential for maintaining the affinity and specificity were preserved while replacing mouse framework with human germline framework to generate humanized antibody.

The DNA sequences of optimized codons encoding the humanized antibodies were synthesized in GenScript.

Antibody expression and purification: ExpiCHO-S cells (Catl. #A29133, Gibco) transfected with plasmid carrying encoding DNA sequences of indicated antibodies were grown and maintained in the ExpiCHO medium (Catl. #A2910001, Gibco) at 32° C., 5% CO₂, for 12 days. The supernatant was collected after spin at 4000 g for 30 min, and filtered through 0.22 μm membrane. As detailed in the manufacturer manual, antibody bounded with protein A (Catl. #17508001, GE) was washed with 20 mM sodium phosphate (pH7.0) and eluted with 0.1M Glycine (pH3.0). The eluted fraction was neutralized with 0.1M Tris buffer (pH9.0), and then switched to PBS buffer by ultrafiltration centrifugation. The protein concentration was determined with BCA.

Surface plasmon resonance (SPR): kinetics and affinity were determined with Biacore T200. Briefly, recombinant human HER3 antibody was immobilized on the Protein A chip (GE, Cat. #29-1275-55). Antigen at concentrations from 50 nM to a final 0.78125 nM, generated by a 2-fold serial dilution, were run through the chip to determine the affinity and kinetics.

FACS: Cultured cells were digested with 0.25% Trypsin-EDTA, and then spinned at 1500 rpm for 5 mins. The cell pellets were regenerated into 5×10⁶ cells/mL with the FACS solution of PBS containing 5% FBS and 0.2% ProClin300. 50 μL of cell suspension was incubated on ice with 100 μL of primary antibody at the concentration of 1 μg/ml for one hour. Wash twice with the FACS solution. The pellets were regenerated with 100 μL of FACS solution containing Goat anti-mouse IgG-PE (1:1000 dilution) and incubated on ice for one hour in the dark. And then the cells were washed twice and resuspended in 200 μL FACS solution.

Western blot: Proteins separated by SDS-PAGE were transferred to nitrocellulose membrane for western blot. The primary antibodies were listed as the followings: anti-HER2 (Cell Signaling, Catl #:2165S), anti-HER3 (Cell Signaling, Catl #:12708), anti-p-HER3 (Cell Signaling, Catl #:4791), anti-beta-actin (Cell Signaling, Catl #: 4967).

ELISA: Human HER2-ex-huFc, Human HER3-huFc, Human EGFR-his were diluted to 2 μg/mL, 50 μL/well in 96-well plate, incubated at 4° C. overnight. Washed with 0.5×PBST, and then incubated with 100 μL blocking buffer (PBS+3% BSA) at 37° C. for 2 h and washed with 0.5×PBST. 3F8 serially diluted in 1:3 with blocking buffer, was added at 50 μL/well, incubated at 37° C. for 40-50 min and then washed with 0.5×PBST. Goat anti-mouse IgG-HRP (SouthernBiotech, 1030-05) was diluted at 1:20000 with blocking buffer, 50 μL/well, 30 min incubation in the dark and then washed with 0.5×PBST. 50 μL of Luminol buffer A+B mixed at 1:1, was added into each well before detection.

NRG1 induced HER3 phosphorylation: Cells were cultured at 6-well plates and used for experiments when reached 80% confluency. On the day of experiment, cells were washed twice with PBS, and incubated in serum free medium for 6 hours followed by overnight treatment with antibody at 10 μg/mL To induce HER3 phosphorylation, NRG1 was added to the working concentration of 100 ng/mL 30 minutes before cells were harvested for western blot.

Cytotoxicity: Cells were seeded in 96-well plates at 5000/well the day before experiment. 3F8-MMAE was added into cells, to the working concentrations from 100 nM down to 1 pM in a 1:3 dilution, three samples for every single concentration. 72 hours later, cytotoxicity was measured with ATPLite Kit, as indicated in the manufacture's book.

Antibody stress test: purified antibodies at 5 mg/mL are stored at 4° C. as regular practice, incubated at 40° C. for 7 and 14 days for heat stability assessment, regenerated and maintained in pH3.5 glycine solution for 2.4 and 6 hours for acid stability assessment, or goes through repeated freeze and thaw treatment for 4 or 6 cycles for freeze-thaw stability assessment. The aggregation is measured by SEC-HPLC, and binding affinity determined by ELISA.

[⁸⁹Zr]Zr-antibody label: DFO-NCS is conjugated to antibody and label as showed in reference (Zeglis and Lewis, 2015). Briefly, DFO and antibody were mixed at 5:1 molar ratio, and incubated at 37° C. for one hour. DFO-conjugated antibody was purified with SEC-HPLC.

⁸⁹Zr-oxalate solution 0.8 mCi was mixed with DFO-conjugated antibody (0.2 mg/ml) in the HEPES/Na₂CO₂ buffer (pH 7.0-7.5), and incubated at room temperature for 30 minutes. The radiochemical purity was assessed with TLC. Rf is [⁸⁹Zr]Zr-antibody 0-0.3, while Rf of free ⁸⁹Zr is 0.6-1.0.

PET-imaging: Around 100 uCi [⁸⁹Zr]Zr-antibody is administrated intravenously into each animal. Imaging is collected and analyzed with small animal PET-imaging machines at indicated time post injection.

Preparation of 3F8-vc-MMAE and U3-1402: ADC preparation was carried out by CRO. Briefly, the disulfide bonds in the antibodies were reduced with the reagents of TCEP (Tris-2-carboxyethyl-phosphine) and DTPA (Diethylene triamine pentacetate acid) at 25° C. for 1-3 hours. MC-VC-MMAE or MC-GGFG-Dxd was dropwisely added into the reduced antibody solution and incubated at 25° C. for 1-4 hours with gentle agitation. DAR (drug antibody ratio) of 3F8-vc-MMAE was around 3.8, while that of U3-1402 (Patritumab-GGFG-Dxd) was around 8.0. Both drugs were conjugated to the cysteine residues in the antibodies.

The final produce was purified by ultrafiltration. Purity and DAR were assessed with SEC-HPLC and HIC-HPLC respectively.

In vivo efficacy study: Animals were maintained and used according the guidelines of IACUC. BALC/b nude and NPG mice purchased from Charles River and SPF Biotech separately, were housed at 25° C. in a 12-hour dark/light cycle with free access to food and drink. Patient derived xenograft (PDX) models were generated by implanting the cryopreserved tissue fragment subcutaneously. Animals were initiated with drug treatment when tumor size reaching 100-200 mm³ and euthanized at the size of 1000 mm³. Gross heath was observed daily. Tumor size and body weight were monitored and recorded every 3 days.

Software: Data were analyzed with Olinda, GraphPad Prism 6.0 or EXCEL.

### Example 1

Murine 3F8 was incubated with SP2/0 wild type cell or those overexpressing HER3, HER2 or EGFR. The binding intensity was detected with PE-anti-murine secondary antibody in the FACS machine.

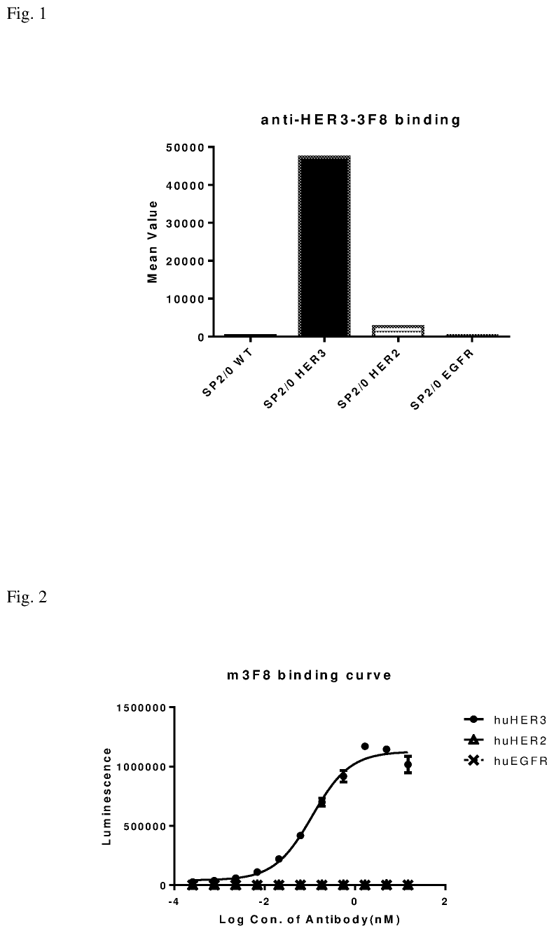

The results were shown in FIG. 1. 3F8 specifically binds SP2/0-HER3 cells, but not others.

### Example 2

The binding affinity of murine 3F8 with human HER3, HER2 and EGFR was determined with ELISA.

The results were shown in FIG. 2. Murine 3F8 only recognizes HER3 but not HER2 or EGFR.

### Example 3

The EC50s of murine 3F8 binding human HER3, HER2 and EGFR were analyzed with GraphPad Prism 6.0. The results were shown in Table 1. 3F8 demonstrated a potent binding affinity at the sub-nanomolar range.

TABLE 1
EC50s of murine 3F8 binding to human HER3, HER2 or EGFR

| 3F8 | huHER3 | huHER2 | huEGFR |
|---|---|---|---|
| EC50 (nM) | 0.11 | NA | NA |
| EC50 (ng/ml) | 16.50 | NA | NA |
| HillSlope | 1.04 | NA | NA |
| R square | 0.99 | NA | NA |

### Example 4

The species selectivity of murine 3F8 against human, monkey, rat and mouse HER3 were determined with ELISA.

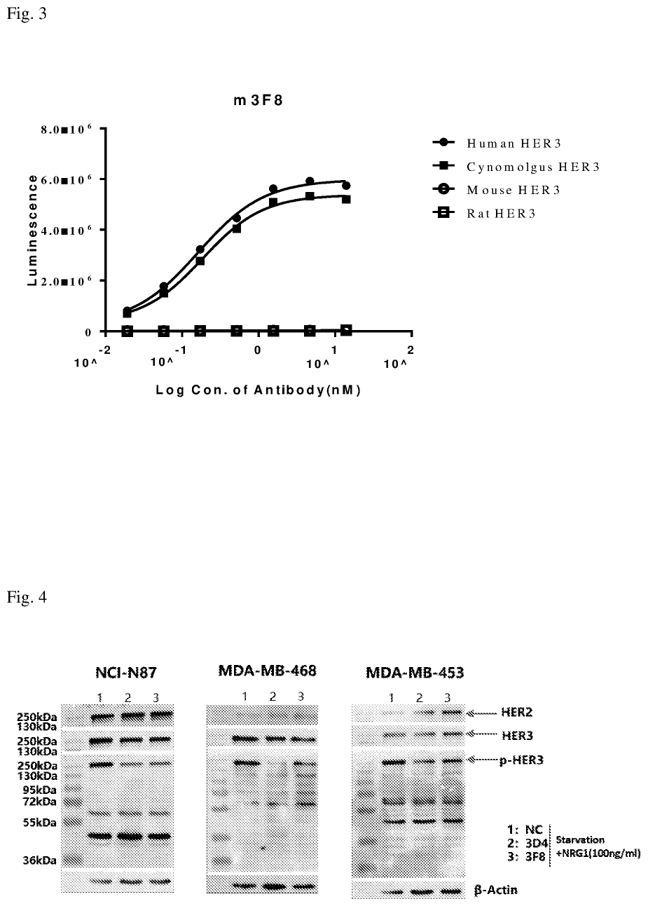

The results were shown in FIG. 3. 3F8 recognized both human and monkey HER3 with similar potency, but not mouse HER3.

### Example 5

The EC₅₀s of murine 3F8 binding to human, monkey, rat and mouse HER3 were analyzed with GraphPad Prism 6.0. The results were shown in Table 2. 3F8 demonstrated equal binding affinity against human and monkey HER3 at the sub-nanomolar range.

TABLE 2
EC50s of murine 3F8 binding to human,
monkey, rat or mouse HER3

| | | Cynomolgus | | |
|---|---|---|---|---|
| | Human HER3 | HER3 | Mouse HER3 | Rat HER3 |
| HillSlope | 1.015 | 1.102 | NA | NA |
| EC₅₀(nM) | 0.1635 | 0.1855 | NA | NA |
| EC₅₀(ng/ml) | 24.525 | 27.825 | NA | NA |
| R square | 0.9957 | 0.9969 | NA | NA |

### Example 6

Murine 3F8 blocked NRG1-induced phosphorylated HER3. NCI-N87, MDA-MB-468 and MDA-MB-453 were treated with NRG1, the HER3 ligand, to induce the downstream phosphorylation of HER3. The effect of murine 3F8 in inhibiting NRG1 induced p-HER3 was determined by western blot. 3D4, a previous proved anti-HER3 antibody that competes the NRG1 binding with HER3 was applied as positive control.

The results were shown in FIG. 4. The data showed that 3F8 reduced phosphorylated HER3 protein level, but had no impact on the protein level of total HER3. Western blot of HER2 also showed that 3F8 had no impact on the HER2 protein level.

### Example 7

Murine 3F8 is rapidly taken up by cells of varying surface level of HER3. Cells of varying level of surface HER3 were incubated with murine 3F8 on the ice as control or at 37° C. for 1 or 4 hours. The internalized fraction was determined by subtracting the cell surface signal of 37° C. incubation from the control of ice incubation.

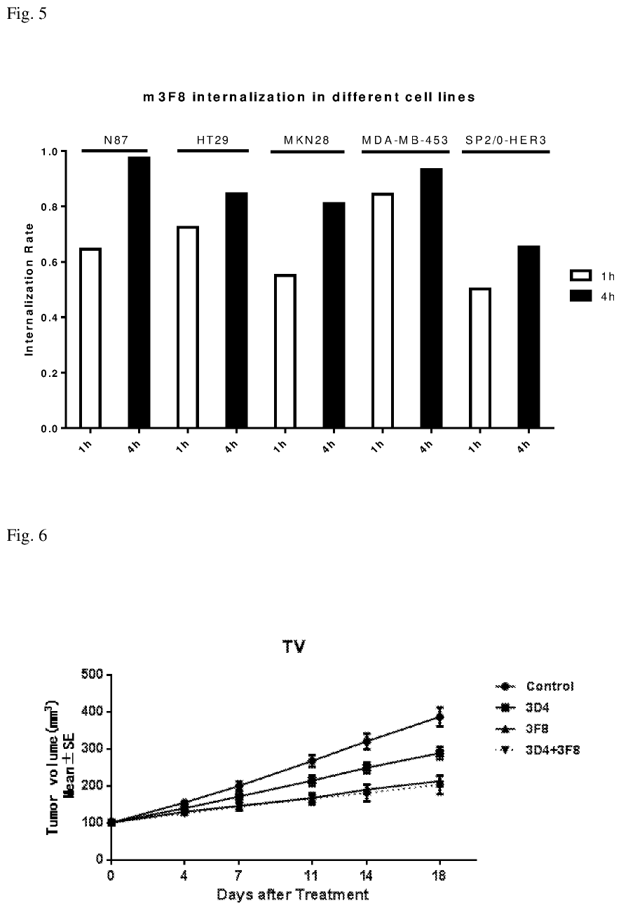

The results were shown in FIG. 5. The data showed that 3F8 was rapidly internalized into the cells. The majority was taken up within one hour incubation and prolonging the incubation time to 4 hours slightly increasing the amount of intracellular fraction, suggesting that the 3F8 endocytosis is a rapid and continuous process.

### Example 8

Anti-HER3 antibodies efficiently inhibited tumor growth in a BT474 subcutaneous xenograft model. Anti-HER3 antibodies m3F8, m3D4 or m3F8+m3D4 combination was intravenously administrated at 25 mg/kg, biweekly for three weeks. Tumor size was monitored every 3-4 days.

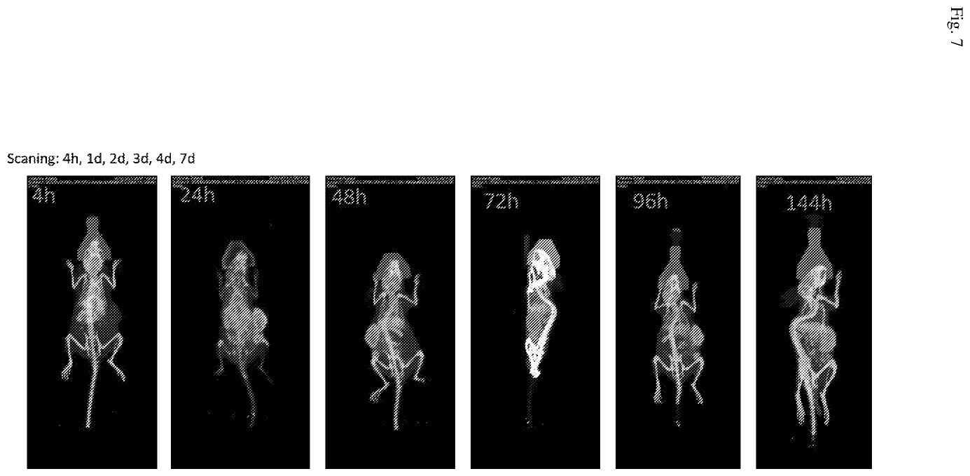

The results were shown in FIG. 6. Both m3F8 and m3D4 significantly inhibit tumor growth (one-way ANOVA, p<0.05), while 3F8 demonstrates a better efficacy. The combination of m3F8 and m3D4 is equally efficient to m3F8 alone. Both 3F8 and 3D4 are anti-HER3 antibodies. m3F8 indicates a murine antibody.

### Example 9

[⁸⁹Zr]Zr-ch3F8 was used to image a gastric PDX model GAS078. [⁸⁹Zr]Zr-ch3F8 was intravenously injected into the gastric model GAS078. Images were collected at 4, 24, 48, 72, 96 and 168 hours after injection. Radio uptake in every organ was analyzed with Olinda and presented in % ID/g (percentage of injection dose/gram tissue).

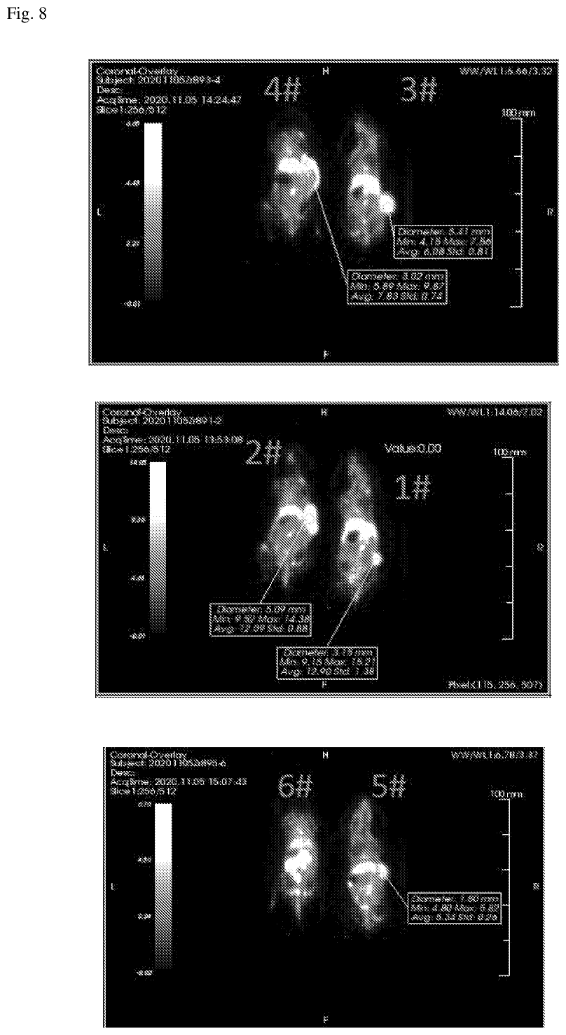

FIG. 7 was a representative imaging of [⁸⁹Zr]Zr-ch3F8 in GAS078 model. Ch3F8 indicates chimeric 3F8 antibody. The data showed that there was a gradually-increased uptake of [⁸⁹Zr]Zr-ch3F8 in the tumor as time progressed. The tumor uptake remained plateau till 96 hour post injection and followed by a slight decrease at 168 hour post injection.

The data were also showed in Table 3. Uptake of [⁸⁹Zr]Zr-ch3F8 in tumor and major organs of heart, liver, kidney and spleen was evaluated in % ID/g (% injection dose/gram tissue).

TABLE 3
Radio uptake in tumor and major organs

| % ID/g | | Heart | Liver | Kidney | spleen | Tumor |
|---|---|---|---|---|---|---|
| 4 h | mice 1 | 23.03 | 20.37 | 11.64 | 15.28 | 5.26 |
| | mice 2 | 14.75 | 14.97 | 8.77 | | 5.38 |
| 24 h | mice 1 | 12.39 | 10.59 | 7.94 | 7.84 | 10.33 |
| | mice 2 | 8.66 | 8.41 | 6.41 | 9.74 | 10.31 |
| 48 h | mice 1 | 6.02 | 8.36 | 6.72 | 10.69 | 12.15 |
| | mice 2 | 3.44 | 6.77 | 6.31 | 5.15 | 11.33 |
| 72 h | mice 1 | 3 | 8.28 | 3.51 | 12.13 | 12.67 |
| | mice 2 | 2.19 | 7.09 | 4.19 | 12.64 | 10.86 |
| 96 h | mice 1 | 1.61 | 6.94 | 3.42 | 11.49 | 12.07 |
| | mice 2 | 1.44 | 6.93 | 2.81 | 10.89 | 8.2 |
| 168 h | mice 1 | 1.09 | 8.44 | 2.89 | 5.68 | 9.16 |
| | mice 2 | 0.81 | 7.18 | 1.94 | 10.88 | 6.18 |

### Example 10

[⁸⁹Zr]Zr-ch3F8 was used to image multiple PDX models. [⁸⁹Zr]Zr-ch3F8 was intravenously injected. Images were collected at 72 hours after injection. Representative images of [⁸⁹Zr]Zr-ch3F8 imaging in 6 animal models were showed in FIG. 8. HER3 expression level in the tumor tissues determined by ELISA and radio-uptake in major organs and tumor tissue 72 hours after injection were listed in Table 4. There was a decent amount of tumor uptake in all 6 tested PDX models.

TABLE 4
HER3 expression level (ng/mg) in the tumor tissues and
radio-uptake in major organs and tumor tissue (% ID/g)

| | | HER3 | | | | | |
|---|---|---|---|---|---|---|---|
| | | (ng/mg, | | | | | |
| No. | Model | Elisa) | heart | kidney | liver | spleen | Tumor |
| 1 | 175P6 | 9.78 | 4.80 | 5.52 | 15.12 | 19.24 | 14.10 |
| 2 | 078P7 | 11.61 | 3.09 | 4.29 | 12.78 | 14.19 | 12.29 |
| 3 | 194P5 | 4.38 (P3) | 7.37 | 5.56 | 5.48 | 15.42 | 14.92 |
| 4 | 142P7 | 0.05 (P6) | 4.15 | 4.13 | 4.93 | 13.09 | 18.27 |
| 5 | 143P6 | 6.07 | 7.90 | 8.30 | 13.64 | 18.01 | 15.48 |
| 6 | 176R | 13.15 | 5.12 | 31.61 | 11.89 | 9.03 | 5.37 |

### Example 11

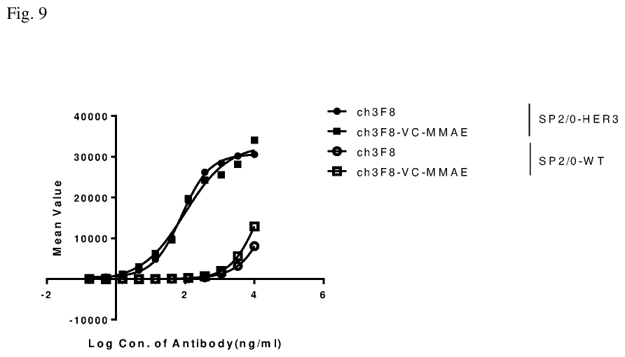

ch3F8-MMAE maintains the similar binding affinity to ch3F8. ch3F8-MMAE binding affinity with HER3 was measured with SP2/0-HER3 by FACS. Results (FIG. 9) showed that ch3F8-MMAE has the same binding affinity as ch3F8 towards SP2/0-HER3, with little binding with HER3 negative SP2/0.

### Example 12

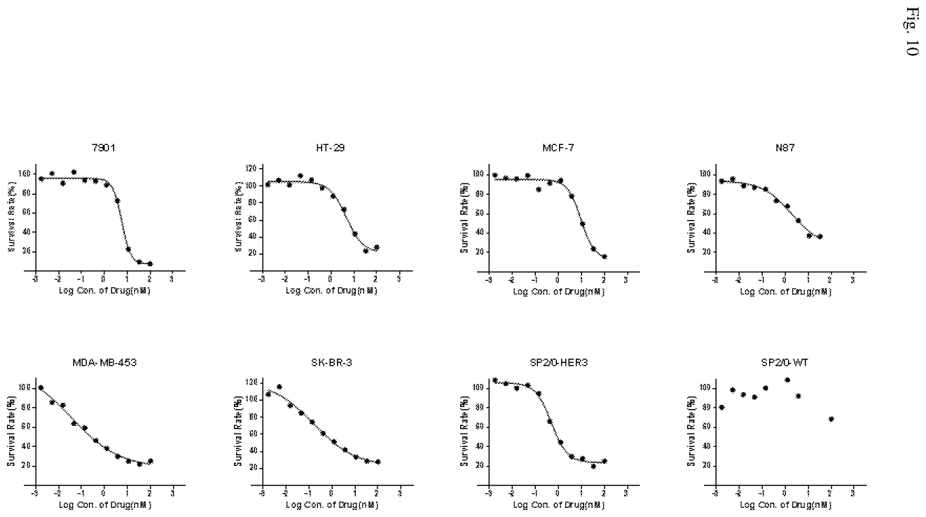

Cytotoxicity of ch3F8-MMAE. Cytotoxicity of ch3F8-MMAE was measured in multiple cell lines with ATPlite. Cells were treated with varying concentration of ch3F8-MMAE for 5 days. The cytotoxicity of ch3F8-MMAE was determined by ATPlite. The data (FIG. 10) showed that ch3F8-MMAE has a strong cytotoxicity in killing HER3(+) cells of 7901, HT-29, MCF-7, N87, MDA-MB-453, SK-BR-3, SP2/0-HER3, but has little effect on HER3(−) cell SP2/0-WT. The cytotoxicity IC₅₀s in killing each tumor cells as showed in FIG. 10 were listed in table 5. Data were analyzed with GraphPad Prism 6.0.

TABLE 5
IC₅₀s of ch3F8-MMAE on multiple cell lines.

| | 7901 | HT-29 | MCF-7 | N87 | MDA-MB-453 | SK-BR-3 | SP2/0-HER3 | SP2/0-WT |
|---|---|---|---|---|---|---|---|---|
| HillSlope | −2.25 | −1.191 | −1.486 | −0.6763 | −0.4233 | −0.4713 | −1.261 | NA |
| IC₅₀ (nM) | 5.744 | 4.672 | 9.494 | 1.907 | 0.04009 | 0.1588 | 0.4864 | NA |
| R square | 0.9916 | 0.9851 | 0.984 | 0.9869 | 0.9897 | 0.9859 | 0.9943 | NA |

### Example 13

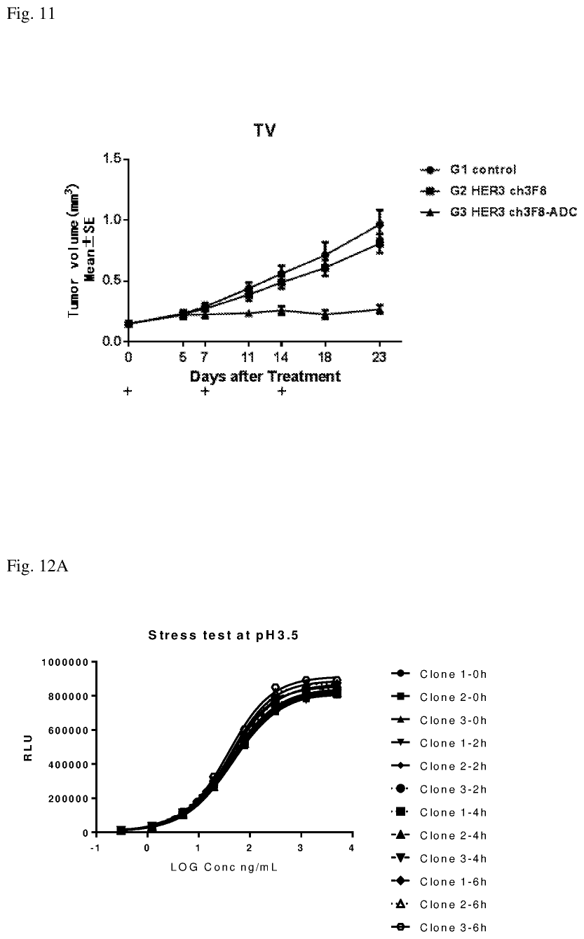

ch3F8-MMAE inhibits tumor growth in the gastric model GAS078. Ch3F8-MMAE was intravenously administrated at 3 mg/kg once every week for three weeks, the tumor inhibition effect was measured every 3-4 days and continued to 10 days after the last dosing. Antibody 3F8 was conducted parallelly, and saline was used as vehicle control. The results were showed in FIG. 11.

### Example 14

Binding affinity of humanized 3F8 (hu3F8) and chimeric 3F8. Binding kinetics of three clones of humanized 3F8 and chimeric 3F8 was determined by Biacore. The three humanized 3F8 clones (Clone1, Clone 2 and Clone 3) have different heave chains while sharing the same heave chain CDRs and one light chain. The amino acid sequences of the heave chains and the light chain of the three humanized 3F8 clones and their encoding DNA sequences are listed herein below. Clone 3 was used as hu3F8 in the Examples below, unless otherwise specified.

All the data listed in Table 6 were processed using the Biacore T200 Evaluation software version 3.1.

TABLE 6
Binding affinity of humanized 3F8 (hu3F8) and chimeric 3F8

| | | | | | Rmax | Chi² |
|---|---|---|---|---|---|---|
| Ligand | Analyte | ka (1/Ms) | kd (1/s) | KD (M) | (RU) | (RU²) |
| Chimeric | His- | 6.28E+05 | 3.91E−04 | 6.24E−10 | 103.1 | 8.35E−02 |
| | HER3 | | | | | |
| Clone 1 | His- | 5.60E+05 | 6.12E−04 | 1.09E−09 | 110.1 | 9.52E−02 |
| | HER3 | | | | | |
| Clone 2 | His- | 6.66E+05 | 3.28E−04 | 4.92E−10 | 113.2 | 4.63E−02 |
| | HER3 | | | | | |
| Clone 3 | His- | 6.44E+05 | 3.48E−04 | 5.40E−10 | 124 | 3.60E−02 |
| | HER3 | | | | | |

### Example 15

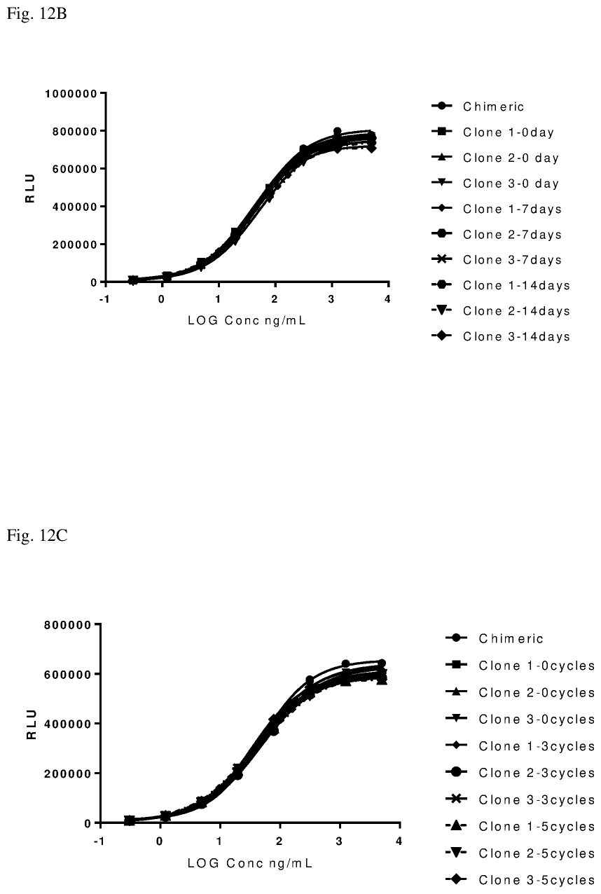

The three clones of hu3F8 were incubated at pH3.5 for 0, 2, 4 and 6 hours and then proceeded to ELISA assay to measure the binding affinity. The results (FIG. 12A) showed that acid treatment has little impact on binding affinity.

EC₅₀s of the three clones of hu3F8 after incubated at pH3.5 for 0, 2, 4 and 6 hours. EC₅₀s were measured by ELISA. The results (Table 7) showed that acid treatment has little impact on binding affinity.

TABLE 7
EC₅₀s of the three clones of hu3F8 after incubated
at pH 3.5 for 0, 2, 4 and 6 hours

| Incubation time | EC₅₀ ng/ml | | |
|---|---|---|---|
| (hr) | Clone 1 | Clone 2 | Clone 3 |
| 0 | 38.74 | 45.04 | 38.71 |
| 2 | 38.11 | 48.31 | 41.71 |
| 4 | 40.89 | 44.54 | 39.45 |
| 6 | 38.96 | 41.18 | 38.06 |

Three clones of hu3F8 were incubated at 40° C. for varying days and then proceeded to ELISA assay to measure the binding affinity. Chimeric antibody was measured parallelly. The results were showed in FIG. 12B.

EC₅₀s of three clones of hu3F8 incubated at 40° C. for varying days. Three clones of humanized 3F8 antibodies were incubated in saline at 40° C. for 7 or 14 days, and binding affinity after this heat stress test was determined by ELISA. Chimeric 3F8 was measured parallelly. The results (Table 8) showed that heat stress has little impact on binding affinity.

TABLE 8
EC₅₀s of three clones of hu3F8 incubated
at 40° C. for varying days.

| Time (hr) | EC₅₀ ng/mL | | | |
|---|---|---|---|---|
| incubated at 40° C. | Chimeric | Clone 1 | Clone 2 | Clone 3 |
| 0 | 47.37 | 39.16 | 45.40 | 42.44 |
| 7 | / | 42.35 | 48.09 | 46.96 |
| 14 | / | 41.00 | 52.03 | 41.25 |

Three clones of hu3F8 were frozen and thaw for 3 or 5 cycles and then proceeded to ELISA assay to measure the binding affinity. Chimeric antibody was measured parallelly. The results (FIG. 12C) showed that repeated freeze-thaw has little impact on binding affinity.

EC₅₀s of three clones of hu3F8 gone through multiple cycles of freeze and thaw stress test were also showed in Table 9.

TABLE 9
EC₅₀s of three clones of hu3F8 gone through multiple
cycles of freeze and thaw stress test

| | |
|---|---|
| Cycles of freeze | EC₅₀ ng/mL |

| and thaw | Chimeric | Clone 1 | Clone 2 | Clone 3 |
|---|---|---|---|---|
| 0 | 47.06 | 45.41 | 42.46 | 42.25 |
| 3 | / | 44.29 | 46.66 | 37.42 |
| 5 | / | 38.58 | 43.79 | 33.63 |

Together, these data, hu3F8 maintains binding affinity after stress test of heat, acid and repeat free-thaw, demonstrates that hu3F8 has a good developability profile.

### Example 16

Aggregation assessment of three clones of the hu3F8 after stress test and their counterparts stored at 4° C. Aggregation of three hu3F8 antibodies after acid treatment, repeated freeze and thaw and incubation at 40° C. was measured with SEC-HPLC, compared with each antibody stored at 4° C. The results were listed in Table 10. All clones maintained large than 95% of monomer after stress test, suggesting little trend of aggregation.

TABLE 10
Aggregation assessment results of three clones of the hu3F8 after stress test

| | Clone 1 | | | Clone 2 | | | Clone 3 | | |
|---|---|---|---|---|---|---|---|---|---|
| test items | Monomers | Dimers | Tetramers | Monomers | Dimers | Tetramers | Monomers | Dimers | Tetramers |
| Retention | 8.84-8.85 | 7.33-7.35 | 6.66-6.68 | 8.86-8.87 | 7.36-7.37 | 6.70-6.73 | 8.86-8.87 | 7.36-7.37 | 6.70-6.73 |
| time | | | | | | | | | |
| antibodies | 94.68% | 4.36% | 0.96% | 96.67% | 3.16% | 0.17% | 97.10% | 2.90% | — |
| stored at 4° C. | | | | | | | | | |
| 40° C. 7 d | 95.02% | 4.22% | 0.76% | 96.94% | 3.03% | 0.03% | 97.09% | 2.91% | — |
| 40° C. 14 d | 95.03% | 4.22% | 0.75% | 96.44% | 3.39% | 0.17% | 97.04% | 2.96% | — |
| pH3.5 2 h | 94.63% | 4.41% | 0.96% | 96.98% | 2.99% | 0.04% | 97.04% | 2.96% | — |
| pH3.5 4 h | 95.06% | 4.22% | 0.73% | 96.52% | 3.27% | 0.22% | 97.13% | 2.87% | — |
| pH3.5 6 h | 94.80% | 4.29% | 0.91% | 96.57% | 3.27% | 0.16% | 97.06% | 2.94% | — |
| freeze and | 94.63% | 4.41% | 0.96% | 96.65% | 3.22% | 0.13% | 96.97% | 2.90% | 0.13% |
| thaw 3 cycles | | | | | | | | | |
| freeze and | 94.69% | 4.43% | 0.88% | 96.31% | 3.44% | 0.25% | 97.01% | 2.87% | 0.12% |
| thaw 5 cycles | | | | | | | | | |

### Example 17

PTM (post-translational modification) analysis of hu3F8. P1: hu3F8 no stress; P2: hu3F8 stress at 40° C. for 2 weeks. Hu3F8 without stress or with stress at 40° C. for 2 weeks were digested with trypsin and post-translational modification was subsequentially analyzed with mass spectrometry. The data (Table 11) showed that there is a slightly increase of deamidation at HC:372-393. In general, there is not big difference in PTM before and after stress, suggesting a good developability profile.

TABLE 11
PTM analysis results of hu3F8

| | | | Retention | | | |
|---|---|---|---|---|---|---|
| | Peptide | | time | Post-translational | EIC ratio (%) | |
| Series | fragment | Peptide sequence | (min) | modification | P1 | P2 |
| 1 | HC:1-13 | QVQLQESGPGLVK | 29.44 | / | / | / |
| | | QVQLQESGPGLVK | 37.65 | pE (Q) | 99.4 | 99.7 |
| 2 | HC:58-65 | NNFNPSLK | 22.99 | / | | |
| | | NNFNPSLK | 24.67 | Deamidation (N) | 1.1 | 1.1 |
| 3 | HC:250-256 | DTLMISR | 25.38 | / | | |
| | | DTLMISR | 22.07 | Oxidation (M) | 22 | 3.1 |
| 5 | HC:298-313 | VVSVLTVVHQDWLNGK | 62.21 | / | / | / |
| | | VVSVLTVVHQDWLNGK | 63.86 | Deamidation (N) | 2.6 | 2.7 |
| | | | 63.55 | | | |
| | | VVSVLTVVHQDWLNGK | 65.32 | Succinimide | 3.9 | 3.9 |
| | | | | formation (N) | | |
| 6 | HC:372-393 | GFYPSDIAVEWESNGQPENNYK | 50.8 | / | / | / |
| | | GFYPSDIAVEWESNGQPENNYK | 51.73 | Deamidation (N) | 22 | 5.8 |
| | | GFYPSDIAVEWESNGQPENNYK | 51.55 | | | |
| 7 | HC:418-440 | WQQGNYFSCSVMHEALHNHYTQK | 37.78 | / | / | / |
| | | WQQGNVFSCSVMHEALHNHYTQK | 38.52 | Deamidation (N) | 1.8 | 1.9 |
| 8 | HC:441-448 | SLSLSPGK | 23.18 | / | / | / |
| | | SLSLSPGK | 27.46 | -K | 96.1 | 96.0 |
| 9 | LC:127-142 | SGTASVVCLLNNFYPR | 64.43 | / | / | / |
| | | SGTASVVCLLNNFYPR | 53.82 | Deamidation (N) | 0.9 | 1.0 |
| | | | 67.25 | | | |

### Example 18

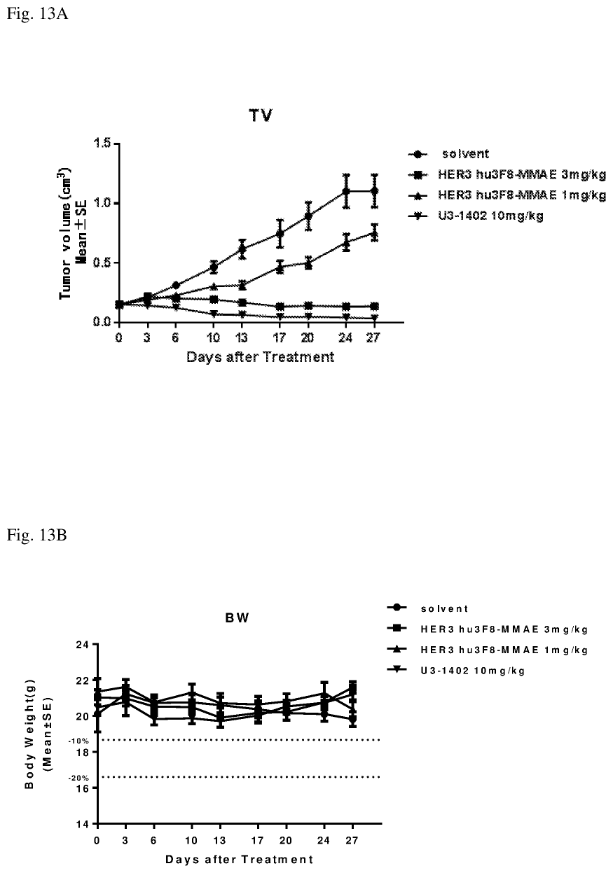

hu3F8-MMAE dose dependently inhibits tumor growth in the gastric PDX model GAS078. Hu3F8-MMAE was administrated at 1, 3 mg/kg intravenously once a week for four weeks. U3-1402 was conducted parallelly at 10 mg/kg. Tumor size and body weight were monitored every 3-4 days. The data demonstrates that hu3F8-MMAE dose-dependently inhibits tumor growth (FIG. 13A) and has little impact on the body weight (FIG. 13B). Hu3F8-MMAE at 3 mg/kg is comparable to U3-1402 at 10 mg/kg in terms of tumor inhibition.

### Example 19

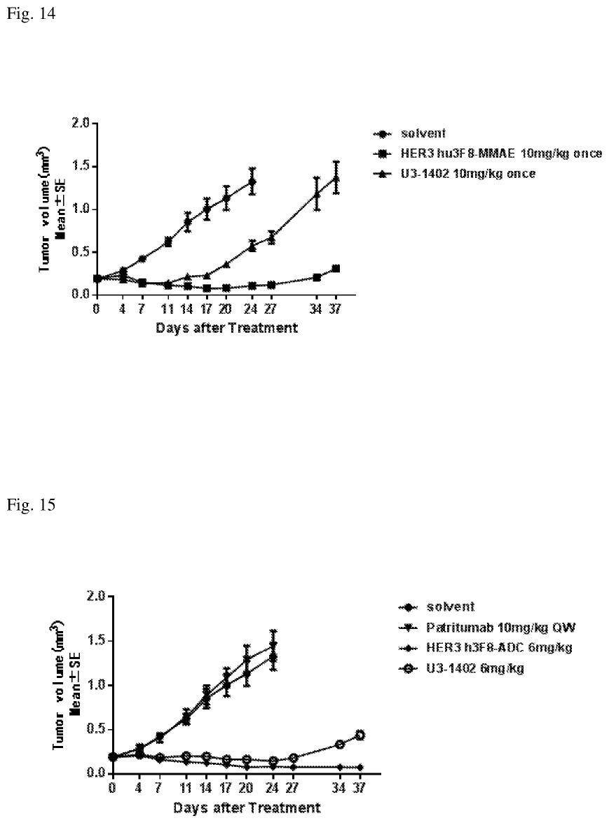

hu3F8-MMAE inhibits tumor growth in the gastric PDX model GAS078 at 10 mg/kg with a single shot. U3-1402, an ADC targeting HER3 via anti-HER3 antibody patritumab was conducted parallelly. Both hu3F8-MMAE and U3-1402 were administrated at 10 mg/kg once, and the tumor inhibition was monitored every 3-4 days. The data (FIG. 14) showed that hu3F8-MMAE at 10 mg/kg is more potent than U3-1402 at 10 mg/kg in inhibiting tumor growth in the GAS078 model.

### Example 20

hu3F8-MMAE inhibits tumor growth in the gastric PDX model GAS078 at 6 mg/kg. U3-1402 is an ADC targeting HER3 via anti-HER3 antibody patritumab. Hu3F8-MMAE was administrated at 6 mg/kg intravenously once a week for three weeks. Patritumab, the anti-HER3 antibody part of U3-1402, and U3-1402 was conducted parallelly at 10 mg/kg, once a week for three weeks. The observation was continued to 37 days, 16 days after the last dosing. Tumor size and body weight were monitored every 3-4 days. The data (FIG. 15) demonstrates that hu3F8-MMAE inhibits tumor growth equally potent to U3-1402 during the drug administration but has a more lasting efficacy.

### Example 21

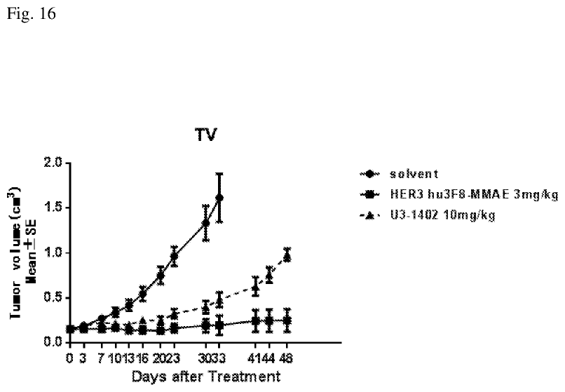

hu3F8-MMAE inhibits tumor growth in the colorectal PDX model CS226. Three groups of animals were treated with solvent, U3-1402 at 10 mg/kg and hu3F8-MMAE at 3 mg/kg respectively for three weeks. Tumor inhibition was measured every three days. Both U3-1402 and hu3F8-MMAE significantly inhibit tumor growth, compared to the solvent treated group. Additionally, hu3F8 at 3 mg/kg is not inferior to U3-1402 at 10 mg/kg (FIG. 16).

Here we report a new anti-HER3 antibody or ADC molecule that efficiently inhibits tumor cell growth in vitro and in vivo, with reasonable safety margin. A HER3 antibody, 3F8, was identified from the mouse hybridoma immunized with SP2/0 cells overexpressing human HER3. 3F8 recognizes human and monkey HER3 with a sub-nanomolar binding affinity, and a high selectivity against the other ERBB family members. Additionally, it is quickly and efficiently up-taken by cells with varying levels of HER3, a property deemed essential for a desirable ADC drug. A PET-imaging study showed that [⁸⁹Zr]Zr-3F8 is significantly accumulated in the tumors of PDX models, indicating that 3F8 could be a highly efficient vehicle carrying cytotoxicity into the tumor cells. As such, a MMAE-conjugated 3F8 was generated and tested for tumor inhibition efficacy. In the in vitro assessment, 3F8-MMAE selectively kills HER3 expressing tumor cells, with IC₅₀ around 1 nM, subject to sensitivity to MMAE, while leaves the HER3 negative cells untouched. Efficacy study showed that 3F8-MMAE dose-dependently inhibits tumor growth, with no gross impact on body weight and no observable hematotoxicity. The humanized version of 3F8, hu3F8, and hu3F8-MMAE maintain the abovementioned features of binding affinity, selectivity, and tumor inhibition in PDX model, equivalent to U3-1402. Hu3F8 has a good developable profile, with little change in binding affinity, aggregation and post-translational modification in the stress tests of repeated freeze-thaw treatment, acid incubation and storage at 40° C. Taken together, 3F8 or its ADC hu3F8-MMAE is a promising oncology therapeutic approach and may offer an alternative for unmet medical needs in the rising challenges of drug resistance.

Listed below are some amino acid sequences and nucleic acid sequences. Antibody sequence numbering is based on Kabat.

Variable region of murine 3F8 light chain:
Nucleotide acid sequence:
ATGATGTCCTCTGCTCAGTTCCTTGGTCTCCTGTTGCTCTGTTTTCAAGGTACCAGA
TGTGATATCCAGATGACACAGACTACATCCTCCCTGTCTGCCTCTCTGGGAGACAG
AGTCACCATCAGTTGCAGGGCAAGTCAGGACATTAGCAATTATTTAAACTGGTATC
AGCAGAAACCAGATGGCACTTTTAAACTCCTGATCTACTACACATCAATATTACAC
TCAGGAGTCCCATCAAGGTTCAGTGGCAGTGGGTCTGGAACAGATTATTCTCTCAC
CATTAGCAACCTGGAGCAAGAGGATATTGCCACTTACTTTTGCCAACAGGGTGATA
CGCTTCCTCCCACGTTCGGTGCTGGGACCAAGCTGGAGCTGAAA (SEQ ID NO: 1)
Amino acid sequence:
MMSSAQFLGLLLLCFQGTRCDIQMTQTTSSLSASLGDRVTISCRASQDISNYLNWYQQ
KPDGTFKLLIYYTSILHSGVPSRFSGSGSGTDYSLTISNLEQEDIATYFCQQGDTLPPTFG
AGTKLELK (SEQ ID NO: 2)

| Region | Sequence Fragment | Residues | Length |
|---|---|---|---|
| Leader | MMSSAQFLGLLLLCFQGTRC (SEQ ID NO: 3) | 1-20 | 20 |
| LFR1 | DIQMTQTTSSLSASLGDRVTISC (SEQ ID NO: 4) | 21-43 | 23 |
| CDR-L1 | RASQDISNYLN (SEQ ID NO: 5) | 44-54 | 11 |
| LFR2 | WYQQKPDGTFKLLIY (SEQ ID NO: 6) | 55-69 | 15 |
| CDR-L2 | YTSILHS (SEQ ID NO: 7) | 70-76 | 7 |
| LFR3 | GVPSRFSGSGSGTDYSLTISNLEQEDIATYFC | 77-108 | 32 |
| | (SEQ ID NO: 8) | | |
| CDR-L3 | QQGDTLPPT (SEQ ID NO: 9) | 109-117 | 9 |
| LFR4 | FGAGTKLELKR (SEQ ID NO: 10) | 118-128 | 11 |
| | | | 128 |

Variable region of murine 3F8 heavy chain:
Nucleotide sequence:
ATGAAAGTGTTGAGTCTGTTGTACCTGTTGACAGCCATTCCTGGTATCCTGTCTGAT
GTACAACTTCAGGAGTCAGGACCTGGCCTCGTGAAACCTTCTCAGTCTCTGTCTCT
CACCTGCTCTGTCACTGGCTACTCCATCACCAGTGCTTATTACTGGAACTGGATCC
GGCAGTTTCCAGGAGACAAACTGGAATGGATGGGCTACATAAGCTACGACGGTCG
CAATAATTTCAACCCATCTCTCAAAAATCGAATCTCCATCACTCGTGACACATCTA
AGAACCAGTTTTTCCTGAAGTTGAATTCTGTGACTTCTGGGGACACAGCTACATAT
TACTGTGCAAGAGATGGGGATTACGACTACTTTGACTACTGGGGCCAAGGCACCA
CTCTCACAGTCTCCTCA (SEQ ID NO: 11)
Amino acid sequence:
MKVLSLLYLLTAIPGILSDVQLQESGPGLVKPSQSLSLTCSVTGYSITSAYYWNWIRQF
PGDKLEWMGYISYDGRNNFNPSLKNRISITRDTSKNQFFLKLNSVTSGDTATYYCARD
GDYDYFDYWGQGTTLTVSS (SEQ ID NO: 12)

| Region | Sequence Fragment | Residues | Length |
|---|---|---|---|
| Leader | MKVLSLLYLLTAIPGILS (SEQ ID NO: 13) | 1-18 | 18 |
| HER1 | DVQLQESGPGLVKPSQSLSLTCSVTGYSIT | 19-48 | 30 |
| | (SEQ ID NO: 14) | | |
| CDR-H1 | SAYYWN (SEQ ID NO: 15) | 49-54 | 6 |
| HFR2 | WIRQFPGDKLEWMG (SEQ ID NO: 16) | 55-68 | 14 |
| CDR-H2 | YISYDGRNNFNPSLKN (SEQ ID NO: 17) | 69-84 | 16 |
| HFR3 | RISITRDTSKNQFFLKLNSVTSGDTATYYCAR | 85-116 | 32 |
| | (SEQ ID NO: 18) | | |
| CDR-H3 | DGDYDYFDY (SEQ ID NO: 19) | 117-125 | 9 |
| HFR4 | WGQGTTLTVSS (SEQ ID NO: 20) | 126-136 | 11 |
| | | | 136 |

Amino acid sequence of humanized 3F8-heavy chain: (Clone 3)
MGWSCIILFLVATATGVHSQVQLQESGPGLVKPSETLSLTCTVSGYSITSAYYWNWIR
QPFGKGLEWMGYISYDGRNNFNPSLKNRVSISRDTSKNQFSLKLSSVTAADTATYYCA
RDGDYDYFDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLS
LSPGK (SEQ ID NO: 21)
Nucleotide acid sequence of humanized 3F8-heavy chain: (Clone 3)
ATGGGCTGGTCATGCATTATTCTGTTTCTGGTCGCAACTGCTACAGGCGTGCATAG
TCAAGTGCAGCTGCAGGAGAGCGGACCTGGCCTCGTGAAGCCAAGCGAGACACTG
TCTCTGACATGTACCGTGTCCGGCTACAGCATCACCTCCGCCTACTACTGGAACTG
GATCCGGCAGCCTTTTGGCAAGGGCCTGGAATGGATGGGCTACATCAGCTACGAC
GGCAGAAACAACTTCAACCCCAGCCTGAAAAATAGAGTGTCCATCTCTCGGGACA
CCAGCAAGAACCAGTTCAGCCTGAAGCTGAGCAGCGTGACAGCCGCTGATACCGC
CACATACTACTGCGCCAGAGACGGAGATTATGACTACTTCGACTACTGGGGCCAG
GGCACCACCGTCACCGTGTCTAGCGCCAGCACCAAGGGCCCTTCCGTGTTTCCACT
GGCCCCCTCCTCTAAATCCACATCTGGCGGCACCGCCGCCCTGGGCTGTCTGGTGA
AGGACTACTTCCCAGAGCCTGTGACAGTGTCCTGGAACTCTGGCGCCCTGACATCC
GGCGTGCACACATTTCCAGCCGTGCTGCAGAGCTCCGGCCTGTACAGCCTGTCTAG
CGTGGTGACAGTGCCCTCCTCTAGCCTGGGCACACAGACCTATATCTGCAACGTGA
ATCACAAGCCAAGCAATACCAAGGTGGACAAGAAGGTGGAGCCCAAGTCCTGTGA
TAAGACACACACCTGCCCCCCTTGTCCTGCTCCCGAGCTGCTGGGCGGCCCTAGCG
TGTTCCTGTTTCCACCCAAGCCTAAGGACACCCTGATGATCTCCCGGACACCCGAG
GTGACCTGCGTGGTGGTGGACGTGTCTCACGAGGATCCTGAGGTGAAGTTCAACT
GGTATGTGGATGGCGTGGAGGTGCACAATGCCAAGACCAAGCCCAGAGAGGAGC
AGTACAACTCTACATATAGGGTGGTGAGCGTGCTGACCGTGCTGCACCAGGACTG
GCTGAACGGCAAGGAGTATAAGTGCAAGGTGTCCAATAAGGCCCTGCCCGCCCCC
ATCGAGAAGACAATCAGCAAGGCCAAGGGCCAGCCTCGGGAGCCACAGGTGTAC
ACCCTGCCTCCATCCAGAGACGAGCTGACAAAGAACCAGGTGTCTCTGACATGTCT
GGTGAAGGGCTTCTATCCTAGCGATATCGCCGTGGAGTGGGAGTCCAATGGCCAG
CCAGAGAACAATTACAAGACCACACCCCCTGTGCTGGACTCCGATGGCTCCTTCTT
TCTGTATTCCAAGCTGACCGTGGATAAGTCTCGGTGGCAGCAGGGCAACGTGTTCA
GCTGTTCCGTGATGCACGAAGCCCTGCATAATCACTATACTCAGAAATCCCTGTCC
CTGTCACCTGGAAAGTGATAA (SEQ ID NO: 22)
Amino acid sequence of humanized 3F8-light chain: (Clones 1-3)
MGWSCIILFLVATATGVHSDIQMTQSPSSLSASVGDRVTITCRASQDISNYLNWYQQKP
GKAPKLLIYYTSILHSGVPSRFSGSGSGTDYTFTISSLQPEDIATYFCQQGDTLPPTFGGG
TKLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
(SEQ ID NO: 23)
Nucleotide acid sequence of humanized 3F8-light chain: (Clones 1-3)
ATGGGCTGGTCATGTATTATTCTGTTTCTGGTCGCAACTGCTACAGGGGTCCATAG
TGATATTCAGATGACCCAGAGCCCCAGCAGCCTGAGCGCCAGCGTGGGCGATAGA
GTGACCATCACATGTCGGGCCTCTCAGGACATCAGCAACTACCTGAACTGGTATCA
GCAAAAGCCCGGCAAAGCCCCTAAGCTGCTGATCTACTACACCAGCATCCTGCAC
AGCGGAGTGCCATCTAGATTCAGCGGCTCTGGCAGCGGCACCGACTACACATTTA
CCATCTCCTCCCTCCAGCCTGAGGACATCGCTACATACTTCTGCCAGCAGGGCGAC
ACCCTGCCTCCTACCTTCGGCGGCGGAACAAAGCTGGAAATCAAGAGGACAGTGG
CCGCCCCAAGCGTGTTCATCTTTCCCCCTTCCGACGAGCAGCTGAAGTCTGGCACC
GCCAGCGTGGTGTGCCTGCTGAACAACTTCTACCCTCGGGAGGCCAAGGTCCAGT
GGAAGGTGGATAACGCCCTGCAGTCTGGCAATAGCCAGGAGTCCGTGACCGAGCA
GGACTCTAAGGATAGCACATATTCCCTGTCTAGCACCCTGACACTGAGCAAGGCC
GATTACGAGAAGCACAAGGTGTATGCCTGTGAAGTCACCCATCAGGGGCTGTCAT
CACCCGTCACTAAGTCATTCAATCGCGGAGAATGCTGATAA (SEQ ID NO: 24)
Amino acid sequence of humanized 3F8-heavy chain: (Clone 1)
MGWSCIILFLVATATGVHSQVQLQESGPGLVKPSETLSLTCTVSGYSITSAYYWNWIR
QPPGKGLEWIGYISYDGRNNFNPSLKNRVTISVDTSKNQFSLKLSSVTAADTAVYYCA
RDGDYDYFDYWGQGTTVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALHNHYTQKSLS
LSPGK (SEQ ID NO: 25)
Nucleotide acid sequence of humanized 3F8-heavy chain: (Clone 1)
ATGGGCTGGTCATGCATTATTCTGTTTCTGGTCGCAACTGCTACAGGCGTGCATAG
TCAAGTGCAGCTGCAGGAGAGCGGCCCTGGACTGGTGAAGCCTAGCGAGACACTG
TCTCTCACCTGTACCGTGTCTGGCTACAGCATCACCTCCGCCTACTACTGGAACTG
GATCCGGCAGCCTCCAGGCAAGGGCCTGGAATGGATCGGCTACATCAGCTACGAC
GGCAGAAACAACTTCAACCCCAGCCTGAAAAATAGAGTGACCATCTCTGTGGACA
CCAGCAAGAACCAGTTTAGCCTGAAGCTGAGCAGCGTGACAGCCGCTGATACCGC
CGTGTACTACTGCGCCAGAGACGGAGATTATGACTACTTCGACTACTGGGGCCAG
GGCACCACAGTCACAGTGTCCAGCGCCAGCACCAAGGGCCCTTCCGTGTTTCCACT
GGCCCCCTCCTCTAAATCCACATCTGGCGGCACCGCCGCCCTGGGCTGTCTGGTGA
AGGACTACTTCCCAGAGCCTGTGACAGTGTCCTGGAACTCTGGCGCCCTGACATCC
GGCGTGCACACATTTCCAGCCGTGCTGCAGAGCTCCGGCCTGTACAGCCTGTCTAG
CGTGGTGACAGTGCCCTCCTCTAGCCTGGGCACACAGACCTATATCTGCAACGTGA
ATCACAAGCCAAGCAATACCAAGGTGGACAAGAAGGTGGAGCCCAAGTCCTGTGA
TAAGACACACACCTGCCCCCCTTGTCCTGCTCCCGAGCTGCTGGGCGGCCCTAGCG
TGTTCCTGTTTCCACCCAAGCCTAAGGACACCCTGATGATCTCCCGGACACCCGAG
GTGACCTGCGTGGTGGTGGACGTGTCTCACGAGGATCCTGAGGTGAAGTTCAACT
GGTATGTGGATGGCGTGGAGGTGCACAATGCCAAGACCAAGCCCAGAGAGGAGC
AGTACAACTCTACATATAGGGTGGTGAGCGTGCTGACCGTGCTGCACCAGGACTG
GCTGAACGGCAAGGAGTATAAGTGCAAGGTGTCCAATAAGGCCCTGCCCGCCCCC
ATCGAGAAGACAATCAGCAAGGCCAAGGGCCAGCCTCGGGAGCCACAGGTGTAC
ACCCTGCCTCCATCCAGAGACGAGCTGACAAAGAACCAGGTGTCTCTGACATGTCT
GGTGAAGGGCTTCTATCCTAGCGATATCGCCGTGGAGTGGGAGTCCAATGGCCAG
CCAGAGAACAATTACAAGACCACACCCCCTGTGCTGGACTCCGATGGCTCCTTCTT
TCTGTATTCCAAGCTGACCGTGGATAAGTCTCGGTGGCAGCAGGGCAACGTGTTCA
GCTGTTCCGTGATGCACGAAGCCCTGCATAATCACTATACTCAGAAATCCCTGTCC
CTGTCACCTGGAAAGTGATAA (SEQ ID NO: 26)
Amino acid sequence of humanized 3F8-heavy chain: (Clone 2)
MGWSCIILFLVATATGVHSQVQLQESGPGLVKPSETLSLTCTVSGYSITSAYYWNWIR
QPFGKGLEWMGYISYDGRNNFNPSLKNRVTISRDTSKNQFSLKLSSVTAADTAVYYC
ARDGDYDYFDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSL
SLSPGK (SEQ ID NO: 27)
Nucleotide acid sequence of humanized 3F8-heavy chain: (Clone 2)
ATGGGCTGGTCATGCATTATTCTGTTTCTGGTCGCAACTGCTACAGGCGTGCATAG
TCAAGTGCAGCTGCAGGAGAGCGGCCCCGGCCTGGTGAAGCCTAGCGAGACACTG
AGCCTCACCTGTACCGTGTCCGGCTACAGCATCACCAGCGCCTACTACTGGAACTG
GATCCGGCAGCCTTTTGGCAAGGGCCTGGAATGGATGGGCTACATCTCCTACGAC
GGCAGAAACAACTTCAACCCATCTCTGAAAAATAGAGTGACCATCAGCCGGGACA
CAAGCAAGAACCAGTTCAGCCTGAAGCTGTCTAGCGTGACAGCCGCTGATACCGC
CGTGTACTACTGCGCCAGAGACGGAGATTATGACTACTTCGACTACTGGGGACAG
GGCACCACCGTGACAGTCAGCTCTGCCAGCACCAAGGGCCCTTCCGTGTTTCCACT
GGCCCCCTCCTCTAAATCCACATCTGGCGGCACCGCCGCCCTGGGCTGTCTGGTGA
AGGACTACTTCCCAGAGCCTGTGACAGTGTCCTGGAACTCTGGCGCCCTGACATCC
GGCGTGCACACATTTCCAGCCGTGCTGCAGAGCTCCGGCCTGTACAGCCTGTCTAG
CGTGGTGACAGTGCCCTCCTCTAGCCTGGGCACACAGACCTATATCTGCAACGTGA
ATCACAAGCCAAGCAATACCAAGGTGGACAAGAAGGTGGAGCCCAAGTCCTGTGA
TAAGACACACACCTGCCCCCCTTGTCCTGCTCCCGAGCTGCTGGGCGGCCCTAGCG
TGTTCCTGTTTCCACCCAAGCCTAAGGACACCCTGATGATCTCCCGGACACCCGAG
GTGACCTGCGTGGTGGTGGACGTGTCTCACGAGGATCCTGAGGTGAAGTTCAACT
GGTATGTGGATGGCGTGGAGGTGCACAATGCCAAGACCAAGCCCAGAGAGGAGC
AGTACAACTCTACATATAGGGTGGTGAGCGTGCTGACCGTGCTGCACCAGGACTG
GCTGAACGGCAAGGAGTATAAGTGCAAGGTGTCCAATAAGGCCCTGCCCGCCCCC
ATCGAGAAGACAATCAGCAAGGCCAAGGGCCAGCCTCGGGAGCCACAGGTGTAC
ACCCTGCCTCCATCCAGAGACGAGCTGACAAAGAACCAGGTGTCTCTGACATGTCT
GGTGAAGGGCTTCTATCCTAGCGATATCGCCGTGGAGTGGGAGTCCAATGGCCAG
CCAGAGAACAATTACAAGACCACACCCCCTGTGCTGGACTCCGATGGCTCCTTCTT
TCTGTATTCCAAGCTGACCGTGGATAAGTCTCGGTGGCAGCAGGGCAACGTGTTCA
GCTGTTCCGTGATGCACGAAGCCCTGCATAATCACTATACTCAGAAATCCCTGTCC
CTGTCACCTGGAAAGTGATAA (SEQ ID NO: 28)

### REFERENCE

Brian M. Zeglis and Jason S. Lewis. The bioconjugation and radiosynthesis of ⁸⁹Zr-DFO-labeled antibodies. J. Vis. Exp. 2015: (96): 52521.
Kuramochi T et al. Humanization and simultaneous optimization of monoclonal antibody. Methods Mol Biol. 2014: 1060:123-37.

1. An anti-HER3 antibody or an antigen-binding fragment thereof, comprising a heavy chain variable region (VH), wherein the VH comprises:
CDR-H1 comprising the amino acid sequence of SEQ ID NO: 15, CDR-H2 comprising the amino acid sequence of SEQ ID NO: 17 and CDR-H3 comprising the amino acid sequence of SEQ ID NO: 19.

2. The anti-HER3 antibody or an antigen-binding fragment of claim 1, wherein the VH comprises CDR-H1 of SEQ ID NO: 15, CDR-H2 of SEQ ID NO: 17 and CDR-H3 of SEQ ID NO: 19.

3. The anti-HER3 antibody or an antigen-binding fragment of claim 1, further comprising a light chain variable region (VL), wherein the VL comprises:
CDR-L1 comprising the amino acid sequence of SEQ ID NO: 5, CDR-L2 comprising the amino acid sequence of SEQ ID NO: 7 and CDR-L3 comprising the amino acid sequence of SEQ ID NO: 9.

4. The anti-HER3 antibody or an antigen-binding fragment of claim 3, wherein the VL comprises CDR-L1 of SEQ ID NO: 5, CDR-L2 of SEQ ID NO: 7 and CDR-L3 of SEQ ID NO: 9.

5. The anti-HER3 antibody or an antigen-binding fragment of claim 1, wherein the VH comprises the amino acid sequence of SEQ ID NO: 12 with or without a leader sequence of SEQ ID NO: 13, or an amino acid sequence at least 85%, 86%, 87%, 88%, 89%, 90%, 91%, 92%, 93%, 94%, 95%, 96%, 97%, 98%, or 99% identical thereto.

6. The anti-HER3 antibody or an antigen-binding fragment of claim 3, wherein the VL comprises the amino acid sequence of SEQ ID NO: 2 with or without a leader sequence of SEQ ID NO: 3, or an amino acid sequence at least 85%, 86%, 87%, 88%, 89%, 90%, 91%, 92%, 93%, 94%, 95%, 96%, 97%, 98%, or 99% identical thereto.

7. The anti-HER3 antibody or an antigen-binding fragment of claim 1, wherein the antibody is a mouse, chimeric, humanized or human antibody.

8. The anti-HER3 antibody or an antigen-binding fragment of claim 1, wherein the heavy chain of the antibody is of the type of IgG1.

9. The anti-HER3 antibody or an antigen-binding fragment of claim 1, wherein the antibody is a humanized antibody, and the heavy chain comprises the amino acid sequence of SEQ ID NO: 21, 25 or 27 with or without a leader sequence in it, or an amino acid sequence at least 85%, 86%, 87%, 88%, 89%, 90%, 91%, 92%, 93%, 94%, 95%, 96%, 97%, 98%, or 99% identical thereto, wherein the leader sequence comprises an amino acid sequence of amino acid positions 1 to 19 of SEQ ID NO: 21, 25, or 27.

10. The anti-HER3 antibody or an antigen-binding fragment of claim 3, wherein the antibody is a humanized antibody, and the light chain comprises the amino acid sequence of SEQ ID NO: 23 with or without a leader sequence in it, or an amino acid sequence at least 85%, 86%, 87%, 88%, 89%, 90%, 91%, 92%, 93%, 94%, 95%, 96%, 97%, 98%, or 99% identical thereto, wherein the leader sequence comprises an amino acid sequence of amino acid positions 1 to 19 of SEQ ID NO: 23.

11-14. (canceled)

15. An isolated nucleic acid, wherein the isolated nucleic acid comprises a polynucleotide acid sequence encoding the anti-HER3 antibody or antigen-binding fragment of claim 1.

16-17. (canceled)

18. A host cell, wherein the host cell comprises the isolated nucleic acid of claim 15 and expresses the anti-HER3 antibody or antigen-binding fragment encoded by the isolated nucleic acid.

19. (canceled)

20. An antibody conjugate, wherein the antibody conjugate comprises the anti-HER3 antibody or antigen-binding fragment of claim 1 conjugated to a chemical moiety and wherein the antibody conjugate is an antibody drug conjugate (ADC).

21. The antibody conjugate of claim 20, wherein the anti-HER3 antibody or antigen-binding fragment is conjugated to the chemical moiety via a linker.

22-24. (canceled)

25. The antibody conjugate of claim 20, wherein the chemical moiety is a radioactive isotope, a chemotherapeutic agent, or a cytotoxic agent.

26. (canceled)

27. The antibody conjugate of claim 25, wherein the cytotoxic agent is selected from auristatin E, auristatin F, MMAE and MMAF.

28. A pharmaceutical composition, wherein the pharmaceutical composition comprises the anti-HER3 antibody or antigen-binding fragment of claim 1 and a pharmaceutically acceptable carrier.

29-35. (canceled)

36. A method of treating a cancer in a subject, comprising administrating to the subject a therapeutically effective amount of the anti-HER3 antibody or antigen-binding fragment of claim 1, wherein the cancer expresses HER3.

37. (canceled)

38. The method of claim 36, wherein the cancer is a gastric or colorectal cancer.

39. The anti-HER3 antibody or an antigen-binding fragment of claim 1, wherein the VH comprises an amino acid sequence of amino acid positions 20-137 of SEQ ID NO: 21, 25 or 27.

40. The anti-HER3 antibody or an antigen-binding fragment of claim 1, wherein the VL comprises an amino acid sequence of amino acid positions 20-126 of SEQ ID NO: 23.

41. A pharmaceutical composition, wherein the pharmaceutical composition comprises the antibody conjugate of claim 20 and a pharmaceutically acceptable carrier.

42. A method of treating a cancer in a subject, comprising administrating to the subject a therapeutically effective amount of the antibody conjugate of claim 20, wherein the cancer expresses HER3.

43. The method of claim 42, wherein the cancer is a gastric or colorectal cancer.
